(19) United States
(12) Patent Application Publication
(10) Pub. No.: US 2023/0201301 A1
(43) Pub. Date: Jun. 29, 2023

(54) TREM-1 INHIBITOR FOR USE IN THE TREATMENT OF A SUBJECT SUFFERING FROM A CORONAVIRUS INFECTION

(71) Applicant: INOTREM, Vandoeuvre-lès-Nancy (FR)

(72) Inventors: Marc DERIVE, Villers-lès-Nancy (FR); Margarita SALCEDO-MAGGUILLI, Châtillon (FR); Jean-Jacques GARAUD, Paris (FR); Simon LAMBDEN, Cambridgeshire (GB); Aurélie OLIVIER, Clairefontaine-en-Yvelines (FR)

(73) Assignee: INOTREM, Vandoeuvre-lès-Nancy (FR)

(21) Appl. No.: 18/008,245

(22) PCT Filed: Jun. 7, 2021
(86) PCT No.: PCT/EP2021/065194

§ 371 Date: Dec. 5, 2022

(30) Foreign Application Priority Data
Jun. 5, 2020 (EP) 20305603.1

(51) Int. Cl. A61K 38/10; A61P 31/14; A61P 11/00; G01N 33/569

(52) U.S. Cl. CPC A61K 38/10; A61P 31/14; A61P 11/00; G01N 33/56983; G01N 2333/165; G01N 2333/70503

(57) ABSTRACT

An inhibitor of triggering receptor expressed on myeloid cells 1 (TREM-1) for use in the treatment of coronavirus disease 2019 (COVID-19) in a subject in need thereof, in particular in a subject suffering from a severe form and/or a complication of COVID-19. Also, the use of soluble triggering receptor expressed on myeloid cells-1 (sTREM-1) as a marker in a method for identifying a subject suffering from COVID-19 susceptible to respond to a TREM-1 inhibitor and in a method for monitoring the effectiveness of TREM-1 inhibitor administered to a subject suffering from COVID-19.

### FIELD OF INVENTION

The present invention relates to the treatment of a disease caused by a coronavirus infection, such as COVID-19. In particular, the present invention relates to an inhibitor of triggering receptor expressed on myeloid cells-1 (TREM-1) for use in the treatment of a subject suffering from a disease caused by a coronavirus infection, such as COVID-19.

### BACKGROUND OF INVENTION

Coronaviruses (CoVs) are ribonucleic acid (RNA) viruses of the Coronaviridae family, notably characterized by a distinctive morphology as seen with electron microscopy, i.e., a crownlike appearance resulting from club-shaped spikes projecting from the surface of their envelope. Coronaviruses infect mammals and birds and cause a wide range of respiratory, gastrointestinal, neurologic, and systemic diseases.

Human coronaviruses were initially thought to cause only mild respiratory infections in most cases, such as the common cold. Four endemic human CoVs are thus estimated to account for 10% to 30% of upper respiratory tract infections in human adults. However, in recent years, two highly pathogenic coronaviruses causing severe respiratory diseases emerged from animal reservoirs: severe acute respiratory syndrome coronavirus (SARS-CoV), first identified in 2003, and Middle East respiratory syndrome coronavirus (MERS-CoV), first identified in 2012.

In December 2019, the Wuhan Municipal Health Committee, China, identified a new infectious respiratory disease of unknown cause. Coronavirus RNA was quickly identified in some of the patients and in January 2020, a full genomic sequence of the newly identified human coronavirus SARS-CoV-2 (previously known as 2019-nCoV) was released by Shanghai Public Health Clinical Center & School of Public Health, Fudan University, Shanghai, China. The genomic sequence of SARS-COV-2 has 82% nucleotide identity with the genomic sequence of human SARS-CoV (Chan et al., Emerg Microbes Infect. 2020; 9(1):221-236). Moreover, as previously shown for SARS-CoV, SARS-CoV-2 utilizes ACE2 (angiotensin converting enzyme 2) as receptor for viral cell entry (Hoffmann et al., Cell. 2020; 181(2):271-280.e8).

In infected subjects exhibiting symptoms, the disease caused by SARS-COV-2 is termed “coronavirus disease 2019” (COVID-19). COVID-19 is a respiratory illness with a broad clinical spectrum. The majority of affected subjects experience mild or moderate symptoms. COVID-19 generally presents first with symptoms including headache, muscle pain, fatigue, fever and respiratory symptoms (such as a dry cough, shortness of breath, and/or chest tightness). Other reported symptoms include a loss of smell and/or taste. Some subjects develop a severe form of COVID-19 that may lead to pneumonitis and acute respiratory failure. Complications of COVID-19 include thrombotic or thromboembolic complications, pulmonary embolism, cardiovascular failure, renal failure, liver failure and secondary infections. It is estimated that about 5% of subjects suffering from COVID-19 will require hospitalization, with about 15-25% of the hospitalized subjects requiring admission in intensive care unit (ICU). SARS-CoV-2 infection is thought to be asymptomatic or causing little or no clinical manifestations in 30 to 60% of infected subjects.

Global efforts to identify effective treatments for COVID-19 are ongoing. A number of clinical trials have been undertaken to assess the efficacy of drugs. These include, for example, anti-interleukin 6 agents such as tocilizumab or sarilumab, antiviral agents such as remdesivir (under development) and the combination of lopinavir/ritonavir (available for the treatment of human immunodeficiency virus 1 (HIV-1)), and repurposed drugs such as hydroxychloroquine (used for the prevention and treatment of malaria, and also for the treatment of rheumatoid arthritis and lupus erythematosus). Other drugs currently being investigated include, for example, the immunosuppressant baricitinib (olumiant), and monoclonal antibodies directed against the Spike glycoprotein (also known as S glycoprotein) of SARS-CoV-2 such as the combination of bamlanivimab and etesevimab, regdanvimab, the combination of casirivimab and imdevimab, and sotrovimab. However, so far, only remdesivir (under the name Veklury) has been approved for use (both by the European Medicines Agency and by the US Food and Drug Administration).

Therefore, there is still a need for effective treatments for diseases caused by a coronavirus, such as COVID-19 caused by SARS-CoV-2, in particular for severe forms of such diseases.

The present invention relates to an inhibitor of triggering receptor expressed on myeloid cells-1 (TREM-1) for use in the treatment of a disease caused by a coronavirus, such as COVID-19 caused by SARS-CoV-2, in particular of a severe form and/or a complication of a disease caused by a coronavirus, in a subject in need thereof.

### SUMMARY

The present invention relates to triggering receptor expressed on myeloid cells 1 (TREM-1) inhibitor for use in the treatment of a disease caused by a coronavirus infection in a subject in need thereof. In particular, the present invention relates to a TREM-1 inhibitor for use in the treatment of coronavirus disease 2019 (COVID 19) caused by a severe acute respiratory syndrome coronavirus 2 (SARS-CoV-2) infection in a subject in need thereof.

In one embodiment, the subject is suffering from a severe form and/or at least one complication of the disease caused by a coronavirus infection, in particular COVID-19 caused by SARS-CoV-2. In one embodiment, the at least one complication of the disease caused by a coronavirus infection, in particular COVID-19 caused by SARS-CoV-2, is selected from the group consisting of respiratory failure, including respiratory failure requiring oxygen therapy, respiratory failure requiring mechanical ventilation, acute respiratory failure or acute respiratory distress syndrome (ARDS); persistence of respiratory failure including the requirement for prolonged mechanical ventilation, in particular prolonged mechanical ventilation lasting more than 15 days, and failed extubation; secondary infection or superinfection; thrombotic complications (also referred to as thromboembolic complications) including venous and/or arterial thromboembolism, deep venous thrombosis, pulmonary embolism, and cerebrovascular accidents; cardiocirculatory failure (which may also be referred to as cardiovascular failure); renal failure including acute kidney injury (AKI); liver failure; and any combinations thereof.

As mentioned above, in one embodiment, the coronavirus is severe acute respiratory syndrome coronavirus 2 (SARS-CoV-2) and the disease caused by a coronavirus infection is coronavirus disease 2019 (COVID 19).

In one embodiment, the TREM-1 inhibitor is for administration by intravenous infusion at a dose ranging from about 0.1 mg/kg/h to about 3 mg/kg/h, preferably from about 0.3 mg/kg/h to about 1 mg/kg/h.

In one embodiment, the TREM-1 inhibitor is selected from the group consisting of peptides inhibiting the function, activity and/or expression of TREM-1; antibodies directed to TREM-1, soluble TREM-1 (sTREM-1), TREM-1 ligand and/or sTREM-1 ligand; small molecules inhibiting the function, activity and/or expression of TREM-1; siRNAs directed to TREM-1; shRNAs directed to TREM-1; antisense oligonucleotide directed to TREM-1; ribozymes directed to TREM-1; and aptamers directed to TREM-1. In one embodiment, the TREM-1 inhibitor is a peptide inhibiting the function, activity and/or expression of TREM-1 comprising an amino acid sequence selected from the group consisting of SEQ ID NO: 9, SEQ ID NO: 10, SEQ ID NO: 11, SEQ ID NO: 12, and SEQ ID NO: 13, or comprising an amino acid sequence with at least 80% identity with SEQ ID NO: 9, SEQ ID NO: 10, SEQ ID NO: 11, SEQ ID NO: 12, or SEQ ID NO: 13. In one embodiment, the TREM-1 inhibitor is a peptide inhibiting the function, activity and/or expression of TREM-1 comprising an amino acid sequence as set forth in SEQ ID NO: 10 or comprising an amino acid sequence with at least 80% identity with SEQ ID NO: 10.

The present invention also relates to an in vitro method for identifying a subject suffering from a disease caused by a coronavirus infection, such as COVID-19 caused by SARS-CoV-2, in particular from a severe form and/or a complication of a disease caused by a coronavirus infection, such as COVID-19 caused by SARS-CoV-2, susceptible to respond to a triggering receptor expressed on myeloid cells-1 (TREM-1) inhibitor, said method comprising:
measuring the level of soluble TREM-1 (sTREM-1) in a biological sample from the subject; and
comparing the level of sTREM-1 measured in the biological sample from the subject to a reference value.

In one embodiment, the coronavirus is severe acute respiratory syndrome coronavirus 2 (SARS-CoV-2) and the disease caused by a coronavirus infection is coronavirus disease 2019 (COVID-19).

In one embodiment, the TREM-1 inhibitor is a peptide inhibiting the function, activity and/or expression of TREM-1 comprising an amino acid sequence selected from the group consisting of SEQ ID NO: 9, SEQ ID NO: 10, SEQ ID NO: 11, SEQ ID NO: 12, and SEQ ID NO: 13, or comprising an amino acid sequence with at least 80% identity with SEQ ID NO: 9, SEQ ID NO: 10, SEQ ID NO: 11, SEQ ID NO: 12, or SEQ ID NO: 13.

The present invention also relates to a TREM-1 inhibitor for use in the treatment of a disease caused by a coronavirus infection, such as COVID-19 caused by SARS-CoV-2, in a subject in need thereof as described hereinabove, wherein the subject to be treated is identified according to the method as described hereinabove.

The present invention also relates to in vitro method for monitoring the effectiveness of a triggering receptor expressed on myeloid cells-1 (TREM-1) inhibitor administered to a subject suffering from a disease caused by a coronavirus infection, such as COVID-19 caused by SARS-CoV-2, in particular from a severe form and/or a complication of a disease caused by a coronavirus infection, such as COVID-19 caused by SARS-CoV-2, said method comprising:
measuring the level of soluble triggering receptor expressed on myeloid cells-1 (sTREM-1) in a biological sample from the subject; and
comparing the level of sTREM-1 measured in the biological sample from the subject to a reference value, preferably to a personalized reference value of the subject.

In one embodiment, the personalized reference value of the subject is the level of sTREM-1 measured in a sample obtained from the subject before or at the beginning of the administration of the TREM-1 inhibitor.

In one embodiment, the coronavirus is severe acute respiratory syndrome coronavirus 2 (SARS-CoV-2) and the disease caused by a coronavirus infection is coronavirus disease 2019 (COVID-19).

In one embodiment, the TREM-1 inhibitor is a peptide inhibiting the function, activity and/or expression of TREM-1 comprising an amino acid sequence selected from the group consisting of SEQ ID NO: 9, SEQ ID NO: 10, SEQ ID NO: 11, SEQ ID NO: 12, and SEQ ID NO: 13, or comprising an amino acid sequence with at least 80% identity with SEQ ID NO: 9, SEQ ID NO: 10, SEQ ID NO: 11, SEQ ID NO: 12, or SEQ ID NO: 13.

### Definitions

In the present invention, the following terms have the following meanings:

“TREM-1” refers to “triggering receptor expressed on myeloid cells-1” and is also sometimes referred to as CD354. TREM-1 is a membrane-bound glycoprotein receptor belonging to the immunoglobulin (Ig) superfamily that is notably expressed on myeloid cells. TREM-1 activates downstream signaling pathways with the help of an adapter protein called DAP12 (DNAX-activating protein of 12 kDa). TREM-1 comprises three distinct domains: an Ig-like structure (mostly responsible for ligand binding), a transmembrane part and a cytoplasmic tail which associates with DAP12. Unless specified otherwise, the TREM-1 protein has an amino acid sequence as set forth in SEQ ID NO: 1, corresponding to UniProtKB/Swiss-Prot accession number Q9NP99-1, last modified on Oct. 1, 2000 and to UniProtKB accession number Q38L15-1, last modified on Nov. 22, 2005. Several transcripts are known for TREM-1. The transcript commonly referred to as TREM1-201 (transcript ID ensembl ENST00000244709.8) encodes an amino acid sequence as set forth in SEQ ID NO: 1. The transcript commonly referred to as TREM1-202, also known as TREM-1 isoform 2 (ensembl transcript ID ENST00000334475.10) encodes an amino acid sequence as set forth in SEQ ID NO: 2 (corresponding to UniProtKB/Swiss-Prot accession number Q9NP99-2). The transcript commonly referred to as TREM1-207, also known as TREM-1 isoform 3 (ensembl transcript ID ENST00000591620.1) encodes an amino acid sequence as set forth in SEQ ID NO: 3 (corresponding to UniProtKB/Swiss-Prot accession number Q9NP99-3). The transcript commonly referred to as TREM1-204 (ensembl transcript ID ENST00000589614.5) encodes an amino acid sequence as set forth in SEQ ID NO: 4 (corresponding to UniProtKB/Swiss-Prot accession number K7EKM5-1, last modified Jan. 9, 2013).

“sTREM-1”, for “soluble triggering receptor expressed on myeloid cells-1”, refers to a soluble form of TREM-1 lacking the transmembrane and intracellular domains of TREM-1. In one embodiment, sTREM-1 thus corresponds to the soluble form of the extracellular domain of TREM-1. The soluble TREM-1 may be generated by proteolytic cleavage of TREM-1 Ig-like ectodomain from the membrane-anchored TREM-1 by matrix metalloproteinases (Gomez-Pina et al., J Immunol. 2007 Sep. 15; 179(6):4065-73). In one embodiment, sTREM-1 thus corresponds to a truncated TREM-1 shed from the membrane of myeloid cells, in particular from activated myeloid cells. It was also suggested that sTREM-1 results from an alternative splicing of TREM-1 mRNA. A TREM-1 splice variant was characterized in 2015 by Baruah et al. (J Immunol. 2015 Dec. 15; 195(12):5725-31), and was found to be secreted from primary and secondary human neutrophil granules. In one embodiment, sTREM-1 thus corresponds to a TREM-1 splice variant, in particular to the TREM-1 transcript commonly referred to as TREM1-202, also known as TREM-1 isoform 2, encoding an amino acid sequence as set forth in SEQ ID NO: 2.

“TLT-1” refers to “TREM-like transcript-1”. TLT-1 is a receptor, member of the TREM family, exclusively expressed by megakaryocytes and platelets. TLT-1 contains a v-set Ig type-extracellular domain, a transmembrane region and a cytoplasmic tail that comprises an immunoreceptor tyrosine based inhibitory motif (ITIM) and a polyproline-rich domain.

“About” preceding a figure encompasses plus or minus 10%, or less, of the value of said figure. It is to be understood that the value to which the term “about” refers is itself also specifically, and preferably, disclosed.

“Electrochemiluminescence immunoassay (ECLIA)” refers to an immunoassay wherein the detection of the signal is based on electrochemiluminescence, i.e., a form of chemiluminescence in which the light-emitting chemiluminescent reaction is preceded by an electrochemical reaction.

“Identity” or “identical”, when used in the present invention in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”) Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988). Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. \2, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., J. Mol. Biol. 215, 403-410 (1990)). The well-known Smith Waterman algorithm may also be used to determine identity.

“Marker”, in particular “biomarker” or “biological marker” refers to a variable that can be measured in a biological sample from a subject.

“Measuring” or “measurement”, or alternatively “detecting” or “detection”, mean assessing the presence, absence, quantity, or amount (which can be an effective amount) of a given substance, i.e., sTREM-1, within a biological sample from a subject. “Measuring” or “measurement”, or alternatively “detecting” or “detection” as used herein include the derivation of the qualitative or quantitative concentration of said substance, i.e., sTREM-1, within the biological sample and within the subject (e.g., blood concentration or plasma concentration).

“Pharmaceutically acceptable excipient” or “pharmaceutically acceptable carrier” refers to an excipient or carrier that does not produce an adverse, allergic or other untoward reaction when administered to a mammal, preferably a human. It includes any and all solvents, such as, for example, dispersion media, coatings, antibacterial and antifungal agents, isotonic and absorption delaying agents. A pharmaceutically acceptable excipient or carrier refers to a non-toxic solid, semi-solid or liquid filler, diluent, encapsulating material or formulation auxiliary of any type. For human administration, preparations should meet sterility, pyrogenicity, general safety and purity standards as required by the regulatory offices such as the FDA (US Food and Drug Administration) or EMA (European Medicines Agency).

“Respiratory support” refers to any measure administered to a subject in order to compensate for a respiratory distress or failure experienced by the subject. Examples of such measures include oxygen therapy (also called standard oxygen therapy or supplemental oxygen), such as supplemental oxygen by mask, nasal cannula or nasal prongs, positive pressure, high flow nasal oxygen, non-invasive ventilation (NIV) (e.g., occlusive mask); invasive mechanical ventilation (IMV) requiring tracheal intubation and/or tracheostomy; and extracorporeal membrane oxygenation (ECMO). As used herein, “respiratory support” thus encompasses both oxygen therapy and invasive mechanical ventilation (IMV).

“Standard of care” refers to the care routinely provided to a hospitalized subject suffering from of a disease caused by a coronavirus, in particular COVID-19 caused by SARS-CoV-2. Standard of care may include for example at least one of the following: respiratory support as defined hereinabove, vasopressor therapy (such as for example phenylephrine, norepinephrine, epinephrine, vasopressin, and/or dopamine), fluid therapy, antimicrobial therapy, antiviral therapy, cardiovascular support, renal replacement therapy, and sedation.

“Subject” refers to a mammal, preferably a human. According to the present invention, a subject is a mammal, preferably a human, suffering from a disease caused by a coronavirus, in particular from COVID-19 caused by SARS-CoV-2. In one embodiment, the subject is a “patient”, i.e., a mammal, preferably a human, who/which is awaiting the receipt of, or is receiving medical care or was/is/will be the object of a medical procedure, or is monitored for the development of disease caused by a coronavirus, in particular COVID-19 caused by SARS-CoV-2.

“Confirmed laboratory diagnosis of COVID-19” as used herein refers to COVID-19 caused by SARS-CoV-2 confirmed by a laboratory test such as a rRT-PCR (real-time reverse transcription polymerase chain reaction) test allowing to detect the presence of SARS-CoV-2 in a sample from a subject (such as a sample from a nasal swab, a sample from an oropharyngeal swab, a sputum sample, a lower respiratory tract aspirate, a bronchoalveolar lavage, a nasopharyngeal wash/aspirate or a nasal aspirate) or an antibody test (such as an enzyme-linked immunosorbent assay (ELISA)) allowing to detect the presence of antibodies against SARS-CoV-2 in a sample from a subject (such as a blood sample).

“Disease caused by a coronavirus” and “disease caused by a coronavirus infection” are interchangeable and refer to any symptom or set of symptoms induced in a subject by the presence of a coronavirus in the organism of said subject.

“Therapeutically effective amount” or “therapeutically effective dose” refers to the amount or dose or concentration of a TREM-1 inhibitor as described herein that is aimed at, without causing significant negative or adverse side effects to the subject in need of treatment, preventing, reducing, alleviating or slowing down (lessening) one or more of the symptoms or manifestations of a disease caused by a coronavirus, in particular COVID-19 caused by SARS-CoV-2, or a complication of said disease caused by a coronavirus, in particular COVID-19 caused by SARS-CoV-2.

“Treating” or “Treatment” refers to a therapeutic treatment, to a prophylactic (or preventative) treatment, or to both a therapeutic treatment and a prophylactic (or preventative) treatment, wherein the object is to prevent, reduce, alleviate, and/or slow down (lessen) one or more of the symptoms or manifestations of a disease caused by a coronavirus, in particular COVID-19 caused by SARS-CoV-2, or a complication of said disease caused by a coronavirus, in particular COVID-19 caused by SARS-CoV-2, in a subject in need thereof. Symptoms of a disease caused by a coronavirus, in particular COVID-19 caused by SARS-CoV-2, include, without being limited to, a fever and respiratory symptoms such as dry cough and/or breathing difficulties that may require respiratory support (for example supplemental oxygen, non-invasive ventilation, invasive mechanical ventilation, extracorporeal membrane oxygenation (ECMO)). Manifestations of a disease caused by a coronavirus, in particular COVID-19 caused by SARS-CoV-2, also include, without being limited to, the viral load (also known as viral burden or viral titer) detected in a biological sample from the subject. In one embodiment, “treating” or “treatment” refers to a therapeutic treatment. In another embodiment, “treating” or “treatment” refers to a prophylactic or preventive treatment. In yet another embodiment, “treating” or “treatment” refers to both a prophylactic (or preventive) treatment and a therapeutic treatment. In one embodiment, the object of the treatment according to the present invention is to bring about at least one of the following:
an improvement in the clinical status, for example defined as a decrease in the score assessed with an ordinal scale such as the 6 or 7-point ordinal scale as defined hereinafter;
a decrease in the requirement for respiratory support;
an increase of the ratio of partial pressure of arterial oxygen/inspired oxygen fraction (PaO₂/FiO₂ ratio);
a decrease in the requirement for other organ support, such as cardiovascular support and/or renal replacement therapy;
a discharge from the intensive care unit;
a discharge from hospital; and/or
a reduction in the viral load detected in a sample from the subject.

“7-point ordinal scale” as used herein refers to a tool for assessing the clinical status of a subject suffering from a disease caused by a coronavirus, in particular COVID-19 caused by SARS-CoV-2. The 7-point ordinal scale ranges from 1 to 7, with a lower score corresponding to a better clinical status as indicated below:
a score of 1 corresponds to a subject not hospitalized, with no limitations on activities;
a score of 2 corresponds to a subject not hospitalized, with limitations on activities;
a score of 3 corresponds to a subject hospitalized, not requiring supplemental oxygen;
a score of 4 corresponds to a subject hospitalized, requiring supplemental oxygen;
a score of 5 corresponds to a subject hospitalized, on non-invasive ventilation or high flow oxygen devices;
a score of 6 corresponds to a subject hospitalized, on invasive mechanical ventilation or ECMO;
a score of 7 corresponds to death.

“6-point ordinal scale” as used herein refers to a tool for assessing the clinical status of a subject suffering from a disease caused by a coronavirus, in particular COVID-19 caused by SARS-CoV-2. The 6-point ordinal scale ranges from 1 to 6, with a lower score corresponding to a better clinical status as indicated below:
a score of 1 corresponds to a subject not hospitalized;
a score of 2 corresponds to a subject hospitalized, not requiring supplemental oxygen;
a score of 3 corresponds to a subject hospitalized, requiring supplemental oxygen;
a score of 4 corresponds to a subject hospitalized, on non-invasive ventilation or high flow oxygen devices;
a score of 5 corresponds to a subject hospitalized, on invasive mechanical ventilation or ECMO;
a score of 6 corresponds to death.

### DETAILED DESCRIPTION

The present invention relates to an inhibitor of triggering receptor expressed on myeloid cells-1 (TREM-1) for use in the treatment of a disease caused by a coronavirus infection in a subject in need thereof.

In one embodiment, the coronavirus is a human coronavirus. In one embodiment, the coronavirus is an alpha coronavirus or a beta coronavirus, preferably a beta coronavirus.

Examples of alpha coronaviruses include, without being limited to, human coronavirus 229E (HCoV-229E) and human coronavirus NL63 (HCoV-NL63) also sometimes known as HCoV-NH or New Haven human coronavirus. Examples of beta coronaviruses include, without being limited to, human coronavirus OC43 (HCoV-OC43), human coronavirus HKU1 (HCoV-HKU1), Middle East respiratory syndrome-related coronavirus (MERS-CoV) previously known as novel coronavirus 2012 or HCoV-EMC, severe acute respiratory syndrome coronavirus (SARS-CoV) also known as SARS-CoV-1 or SARS-classic, and severe acute respiratory syndrome coronavirus (SARS-CoV-2) also known as 2019-nCoV or novel coronavirus 2019.

In one embodiment, the coronavirus is selected from the group comprising or consisting of HCoV-229E, HCoV-NL63, HCoV-OC43, HCoV-HKU1, MERS-CoV, SARS-CoV-1 and SARS-CoV-2.

In one embodiment, the coronavirus is selected from the group comprising or consisting of MERS-CoV, SARS-CoV-1 and SARS-CoV-2.

In one embodiment, the coronavirus is a MERS coronavirus, in particular MERS-CoV causing Middle East respiratory syndrome (MERS). Thus, in one embodiment, the subject is suffering from MERS caused by MERS-CoV.

In one embodiment, the coronavirus is a SARS coronavirus.

In one embodiment, the coronavirus is SARS-CoV (also referred to as SARS-CoV-1) causing severe acute respiratory syndrome (SARS) or SARS-CoV-2 causing COVID-19. Thus, in one embodiment, the subject is suffering from SARS caused by SARS-CoV (also referred to as SARS-CoV-1) or from COVID-19 caused by SARS-CoV-2.

In one embodiment, the coronavirus is SARS-CoV-2 causing COVID-19. Thus, in one in one embodiment, the subject is suffering from COVID-19 caused by SARS-CoV 2.

As used herein, “SARS-CoV-2” encompasses SARS-CoV-2 as initially identified in Wuhan, China and any variants thereof. Variants of SARS-CoV-2 may differ from each other by the presence of one or more mutation(s) in any of their proteins, including their nonstructural replicase polyproteins and their four structural proteins, known as the S (spike) protein or glycoprotein, the E (envelope) protein, the M (membrane) protein, and the N (nucleocapsid) protein. In particular, variants of SARS-CoV-2 may differ from each other by the presence of one or more mutation(s) in their Spike glycoprotein (also known as S glycoprotein or S protein). The reference sequence of the Spike glycoprotein, consisting of 1273 amino acids, is as set forth in SEQ ID NO: 21, corresponding to UniProtKB accession number P0DTC2, last modified on Apr. 22, 2020.

As indicated by the US Centers for Disease Control and Prevention (CDC), examples of SARS-CoV-2 variants include, without being limited to:
variant B.1.1.7, also known as Alpha (WHO label), VUI—202012/01, VOC-202012/01, 20I/501Y.V1, or colloquially as the “UK variant or British variant or English variant”, comprising the following mutations (based on the sequence SEQ ID NO: 21): 69del, 70del, 144del, N501Y, A570D, D614G, P681H, T716I, S982A, D1118H, and optionally E484K, S494P, and/or K1191N;
variant B.1.351, also known as Beta (WHO label), 20H/501Y.V2 (formerly 20C/501Y.V2), or colloquially as the “South African” variant, comprising the following mutations (based on the sequence SEQ ID NO: 21): D80A, D215G, 241del, 242del, 243del, K417N, E484K, N501Y, D614G, A701V;
variant P.1, also known as Gamma (WHO label), 20J/501Y.V3, or colloquially as the “Brazilian variant”, comprising the following mutations (based on the sequence SEQ ID NO: 21): L18F, T20N, P26S, D138Y, R190S, K417T, E484K, N501Y, D614G, H655Y, T1027I;
variant P.2, also known as Zeta (WHO label) or 20J, a variant first detected in Brazil, comprising the following mutations (based on the sequence SEQ ID NO: 21): E484K, D614G, V1176F, and optionally F565L;
variant B.1.617, also known as 20A/484Q, or colloquially as the “Indian variant”, comprising the following mutations (based on the sequence SEQ ID NO: 21): L452R, E484Q, D614G;
variant B.1.617.1, also known as Kappa (WHO label) or 20A/S:154K, a variant first detected in India, comprising the following mutations (based on the sequence SEQ ID NO: 21): G142D, E154K, L452R, E484Q, D614G, P681R, Q1071H, and optionally T95I;
variant B.1.617.2, also known as Delta (WHO label) or 20A/S:478K, a variant first detected in India, comprising the following mutations (based on the sequence SEQ ID NO: 21): T19R, 156del, 157del, R158G, L452R, T478K, D614G, P681R, D950N, and optionally G142D;
variant B.1.617.3, a variant first detected in India, comprising the following mutations (based on the sequence SEQ ID NO: 21): T19R, G142D, L452R, E484Q, D614G, P681R, D950N;
variant B.1.427, also known as Epsilon (WHO label) or 20C/S:452R, comprising the following mutations (based on the sequence SEQ ID NO: 21): L452R, D614G;
variant B.1.429, also known as 20C/S:452R, comprising the following mutations (based on the sequence SEQ ID NO: 21): S13I, W152C, L452R, D614G;
variant B.1.525, also known as Eta (WHO label) or 20A/S:484K, comprising the following mutations (based on the sequence SEQ ID NO: 21): A67V, 69del, 70del, 144del, E484K, D614G, Q677H, F888L;
variant B.1.526 also known as Iota (WHO label) or 20C/S:484K, comprising the following mutations (based on the sequence SEQ ID NO: 21): T95I, D253G, D614G, and optionally L5F, S477N, E484K, and/or A701V; and
variant B.1.526.1, also known as 20C, comprising the following mutations (based on the sequence SEQ ID NO: 21): D80G, 144del, F157S, L452R, D614G, D950H, and optionally T791I and/or T859N.

Other examples of variants include a variant comprising the mutation D614G in the amino acid sequence of the Spike glycoprotein (based on the sequence SEQ ID NO: 21).

Thus, in one embodiment, the subject is suffering from COVID-19 caused by SARS-CoV-2 or any variant of SARS-CoV-2. In one embodiment, the subject is suffering from COVID-19 caused by a SARS-CoV-2 variant selected from the group comprising or consisting of variant B.1.1.7 (Alpha), variant B.1.351 (Beta), variant P.1 (Gamma), variant P.2 (Zeta), variant B.1.617, variant B.1.617.1 (Kappa), variant B.1.617.2 (Delta) and/or variant B.1.617.3. In one embodiment, the subject is suffering from COVID-19 caused by the SARS-CoV-2 variant B.1.1.7 (Alpha). In one embodiment, the subject is suffering from COVID-19 caused by the SARS-CoV-2 variant B.1.617, or any of the related variants B.1.617.1 (Kappa), B.1.617.2 (Delta) and/or B.1.617.3.

In one embodiment, the present invention relates to a TREM-1 inhibitor for use in the treatment of a disease caused by a coronavirus infection as described hereinabove, in particular COVID-19, in a subject in need thereof suffering from of a severe form and/or one or more complication(s) of said disease. Thus, in one embodiment, the present invention relates to a TREM-1 inhibitor for use in the treatment of a severe form and/or at least one complication of a disease caused by a coronavirus as described hereinabove, in particular COVID-19. In one embodiment, the present invention relates to a TREM-1 inhibitor for use in the treatment of a severe form of a disease caused by a coronavirus as described hereinabove, in particular of COVID-19. In one embodiment, the present invention relates to a TREM-1 inhibitor for use in the prevention of at least one complication caused by a coronavirus as described hereinabove, in particular of COVID-19.

In one embodiment, a severe form of the disease caused by a coronavirus, in particular a severe form of COVID-19, is defined as requiring hospitalization. In one embodiment, a severe form of the disease caused by a coronavirus, in particular a severe form of COVID-19, is defined as requiring admission in ICU.

In one embodiment, a severe form of the disease caused by a coronavirus, in particular a severe form of COVID-19, is defined as requiring respiratory support as defined hereinabove. In one embodiment, the respiratory support is selected from the group comprising or consisting of oxygen therapy (also called standard oxygen therapy or supplemental oxygen), such as supplemental oxygen by mask, nasal cannula or nasal prongs, positive pressure, high flow nasal oxygen, non-invasive ventilation (NIV) (e.g., occlusive mask); invasive mechanical ventilation (IMV) requiring tracheal intubation and/or tracheostomy; and extracorporeal membrane oxygenation (ECMO). In one embodiment, the respiratory support is selected from the group comprising or consisting of non-invasive ventilation (NIV), supplemental oxygen (also called oxygen therapy) by mask or nasal prongs, positive pressure, high flow nasal oxygen; invasive mechanical ventilation (IMV) requiring tracheal intubation and/or tracheostomy; and extracorporeal membrane oxygenation (ECMO). In one embodiment, a severe form of the disease caused by a coronavirus, in particular a severe form of COVID-19, is defined as requiring invasive mechanical ventilation as described hereinabove. In one embodiment, a severe form of the disease caused by a coronavirus, in particular a severe form of COVID-19, is defined as requiring prolonged respiratory support, in particular prolonged invasive mechanical ventilation.

In one embodiment, prolonged respiratory support, in particular prolonged invasive mechanical ventilation, is respiratory support, in particular invasive mechanical ventilation, lasting at least 5, 6, 7, 8, 9, 10, 11, 12, 13, 14, 15, 16, 17, 18, 19, or 20 days, preferably at least 10 days, more preferably at least 15 days. In one embodiment, prolonged respiratory support, in particular prolonged invasive mechanical ventilation, is respiratory support, in particular invasive mechanical ventilation, lasting at least 1, 2, 3, 4, or 5 week(s), preferably at least 2 weeks. In one embodiment, prolonged respiratory support, in particular prolonged invasive mechanical ventilation, is respiratory support, in particular invasive mechanical ventilation, lasting more than 5, 6, 7, 8, 9, 10, 11, 12, 13, 14, 15, 16, 17, 18, 19, or 20 days, preferably more than 15 days. In one embodiment, prolonged respiratory support, in particular prolonged invasive mechanical ventilation, is respiratory support, in particular invasive mechanical ventilation, lasting more than 1, 2, 3, 4, or 5 week(s), preferably more than 2 weeks.

In one embodiment, the one or more complication(s) of the disease caused by a coronavirus, in particular of COVID-19, is selected from the group comprising or consisting of, respiratory failure; persistence of respiratory failure; secondary infection or superinfection; thrombotic complications (also referred to as thromboembolic complications); cardiocirculatory failure (which may also be referred to as cardiovascular failure); renal failure; liver failure. In one embodiment, the complication of the disease caused by a coronavirus, in particular of COVID-19, is selected from the group comprising or consisting of, respiratory failure, including acute respiratory failure or acute respiratory distress syndrome (ARDS); persistence of respiratory failure including the requirement for prolonged mechanical ventilation, in particular prolonged mechanical ventilation lasting more than 15 days, and failed extubation; secondary infection or superinfection; thrombotic complications including venous and/or arterial thromboembolism; pulmonary embolism; cardiocirculatory failure (which may also be referred to as cardiovascular failure); renal failure including acute kidney injury (AKI); liver failure; and any combinations thereof. In one embodiment, the complication of the disease caused by a coronavirus, in particular of COVID-19, is selected from the group comprising or consisting of, respiratory failure, including respiratory failure requiring oxygen therapy, respiratory failure requiring mechanical ventilation, acute respiratory failure or acute respiratory distress syndrome (ARDS); persistence of respiratory failure including the requirement for prolonged mechanical ventilation, in particular prolonged mechanical ventilation lasting more than 15 days, and failed extubation; secondary infection or superinfection; thrombotic complications (also referred to as thromboembolic complications) including venous and/or arterial thromboembolism, deep venous thrombosis, pulmonary embolism, and cerebrovascular accidents; cardiocirculatory failure (which may also be referred to as cardiovascular failure), including cardiac failure and vascular dysfunction; renal failure including acute kidney injury (AKI); liver failure; and any combinations thereof.

In one embodiment, the complication of the disease caused by a coronavirus, in particular of COVID-19, is respiratory failure. In one embodiment, the complication of the disease caused by a coronavirus, in particular of COVID-19, is respiratory failure requiring oxygen therapy, respiratory failure requiring mechanical ventilation, acute respiratory failure and/or acute respiratory distress syndrome (ARDS). In one embodiment, the complication of the disease caused by a coronavirus, in particular of COVID-19, is persistence of respiratory failure, including the requirement for prolonged mechanical ventilation as defined hereinabove, and failed extubation.

In one embodiment, acute respiratory distress syndrome (ARDS) is diagnosed according to the Berlin Definition (ARDS Definition Task Force, Ranieri et al., JAMA. 2012; 307(23):2526-2533.).

In one embodiment, the complication of the disease caused by a coronavirus, in particular of COVID-19, is a secondary infection or superinfection. In one embodiment, secondary infection is diagnosed when the subject shows clinical, laboratory or radiological signs or symptoms of pneumonia or bacteremia, optionally confirmed by positive culture.

In one embodiment, the complication of the disease caused by a coronavirus, in particular of COVID-19, is a thrombotic complication or a thromboembolic complication. In one embodiment, thrombotic complication (also referred to as thromboembolic complication or thromboembolic event) comprises or consists of venous and/or arterial thromboembolism, deep venous thrombosis, pulmonary embolism, and/or cerebrovascular accident.

In one embodiment, the complication of the disease caused by a coronavirus, in particular of COVID-19, is renal failure including acute kidney injury (AKI) and/or liver failure. In one embodiment, acute kidney injury is diagnosed according to the kidney disease improving global outcomes (KDIGO) clinical practice guidelines (Khwaja, Nephron Clin Pract. 2012; 120(4):c179-c184).

In one embodiment, the complication of the disease caused by a coronavirus, in particular of COVID-19, is cardiocirculatory failure (which may also be referred to as cardiovascular failure), in particular cardiac failure and/or vascular dysfunction. In one embodiment, cardiac failure is defined as the presence of one or more of the following: elevated serum levels of troponin, elevated serum levels of brain type natriuretic peptide (BNP), clinical or radiological features of cardiac failure, and/or a requirement for pharmacological or mechanical support of cardiac function.

In one embodiment, vascular dysfunction is defined as one or more of the following: clinical or laboratory features of vasodilatation, low systemic vascular resistance or blood pressure, and/or requirement for vasopressor medications to maintain adequate blood pressure.

In one embodiment, cardiocirculatory failure is diagnosed when the serum levels of troponin are greater than 12 pg/mL or when there is a requirement for vasopressors.

In one embodiment, the subject suffering from a disease caused by a coronavirus as described hereinabove is not hospitalized. In one embodiment, the subject suffering from a disease caused by a coronavirus as described hereinabove is hospitalized.

In one embodiment, the subject suffering from a disease caused by a coronavirus as described hereinabove is hospitalized but does not require admission in intensive care unit (ICU). In one embodiment, the subject suffering from a disease caused by a coronavirus as described hereinabove is hospitalized and requires admission in ICU. In one embodiment, the subject suffering from a disease caused by a coronavirus as described hereinabove is hospitalized in ICU.

In one embodiment, the subject suffering from a disease caused by a coronavirus as described hereinabove is hospitalized and does not require respiratory support. In one embodiment, the subject suffering from a disease caused by a coronavirus as described hereinabove is hospitalized and requires respiratory support.

In one embodiment, the subject suffering from a disease caused by a coronavirus as described hereinabove is hospitalized and requires supplemental oxygen. In one embodiment, the subject suffering from a disease caused by a coronavirus as described hereinabove is hospitalized and requires non-invasive ventilation (NIV). In one embodiment, the subject suffering from a disease caused by a coronavirus as described hereinabove is hospitalized and requires invasive mechanical ventilation. In one embodiment, the subject suffering from a disease caused by a coronavirus as described hereinabove is hospitalized in ICU and requires invasive mechanical ventilation. In one embodiment, the subject suffering from a disease caused by a coronavirus as described hereinabove is hospitalized in ICU and is under invasive mechanical ventilation. In one embodiment, the subject suffering from a disease caused by a coronavirus as described hereinabove is hospitalized in ICU and has been under invasive mechanical ventilation for less than 6 hours, 12 hours, 18 hours, 24 hours, 30 hours, 36 hours, 42 hours, or 48 hours, preferably for less than 48 hours.

In one embodiment, the subject is a male. In one embodiment, the subject is a female.

In one embodiment the subject is an adult. Thus, in one embodiment, the subject is older than 18, 19, 20 or 21 years of age. In one embodiment the subject is a child. Thus, in one embodiment, the subject is younger 18, 17, 16 or 15 years of age.

In one embodiment, the subject is younger than 85, 80, 75, 70, 65 or 60 years of age. In one embodiment, the subject is 85 years old or younger. In one embodiment, the subject is older than 60, 65 or 70 years of age. In one embodiment, the subject is older than 60, 65 or 70 years of age and younger than 85 years of age. In one embodiment, the subject is 60 years old or older. In one embodiment, the subject is 60 years old or older and younger than 85 years old.

In one embodiment, the subject has a ratio of artery partial pressure of oxygen/inspired oxygen fraction (PaO₂/FiO₂ ratio) lower than about 300 mmHg, preferably lower than about 200 mmHg. Accordingly, in one embodiment, the subject has a PaO₂/FiO₂ ratio lower than about 40 kPa, preferably lower than about 26.7 kPa.

In one embodiment, the subject suffers from acute respiratory failure or from acute respiratory distress syndrome (ARDS) associated to the disease caused by a coronavirus as described hereinabove.

In one embodiment, the subject has a baseline score of 6 on the 7-point ordinal scale used for assessing clinical status as defined hereinabove.

In one embodiment, the subject suffers from at least one comorbidity. As used herein, “comorbidity” refers to a disease or condition coexisting in the subject with the disease caused by a coronavirus. Examples of comorbidities that may coexist in the subject with a disease caused by a coronavirus include, without being limited to, asthma, autoimmune or auto-inflammatory diseases or conditions, cardiovascular diseases or conditions, chronic bronchitis, chronic kidney diseases, chronic liver disease, chronic obstructive pulmonary disease (COPD), cystic fibrosis, diabetes, emphysema, high blood pressure, immunodeficiency, malignancy (i.e., cancer), obesity, pulmonary hypertension, and severe respiratory conditions.

In one embodiment, the subject presents at least one comorbidity selected from the group comprising or consisting of asthma, autoimmune or auto-inflammatory diseases or conditions, cardiovascular diseases or conditions, chronic bronchitis, chronic kidney diseases, chronic liver disease, chronic obstructive pulmonary disease (COPD), cystic fibrosis, diabetes, emphysema, high blood pressure, immunodeficiency, malignancy (i.e., cancer), obesity, pulmonary hypertension, and severe respiratory conditions.

In one embodiment, the subject is or has been identified as susceptible to respond to a therapy, in particular to a TREM-1 inhibitor as described herein. In one embodiment, the subject is or has been identified as susceptible to respond to a therapy, in particular to a TREM-1 inhibitor as described herein, with the method of the present invention as described hereinafter.

According to the present invention, a TREM-1 inhibitor is a pharmaceutically active agent able to inhibit TREM-1 function, activity and/or expression.

In one embodiment, the TREM-1 inhibitor is selected from the group comprising or consisting of peptides inhibiting the function, activity and/or expression of TREM-1; antibodies (or antigen-binding fragments thereof) directed to (or against) TREM-1, soluble TREM-1 (sTREM-1), TREM-1 ligand and/or sTREM-1 ligand; small molecules inhibiting the function, activity and/or expression of TREM-1; siRNAs directed to TREM-1; shRNAs directed to TREM-1; antisense oligonucleotide directed to TREM-1; ribozymes directed to TREM-1; and aptamers directed to TREM-1. In one embodiment, the TREM-1 inhibitor is a peptide inhibiting TREM-1 or an anti-TREM-1 antibody or antigen-binding fragment thereof. In one embodiment, the TREM-1 inhibitor is a TLT-1 peptide inhibiting TREM-1 or an anti-TREM-1 antibody or antigen-binding fragment thereof.

Examples of peptides inhibiting the function, activity and/or expression of TREM-1 include, without being limited to, peptides targeting TREM-1 ligand, such as, for example, TLT-1 derived peptides (i.e., TLT-1 peptides).

In one embodiment, the TREM-1 inhibitor is a peptide inhibiting TREM-1 through its binding to TREM-1 ligand.

In one embodiment, the TREM-1 inhibitor is a TLT-1 peptide.

In one embodiment, the TREM-1 inhibitor is a TLT-1 peptide consisting of between 6 and 20 consecutive amino acids (or consisting of 6 to 20 consecutive amino acids) from the human TLT-1 having an amino acid sequence as set forth in SEQ ID NO: 8 (MGLTLLLLLLLGLEGQGIVGSLPEVLQAPVGSSILVQCHYRL QDVKAQKVWCRFLPEGCQPLVSSAVDRRAPAGRRTFLTDLGGGLLQVEMVTL QEEDAGEYGCMVDGARGPQILHRVSLNILPPEEEEETHKIGSLAENAFSDPAGS ANPLEPSQDEKSIPLIWGAVLLVGLLVAAVVLFAVMAKRKQGNRLGVCGRFLS SRVSGMNPSSVVHHVSDSGPAAELPLDVPHIRLDSPPSFDNTTYTSLPLDSPSGK PSLPAPSSLPPLPPKVLVCSKPVTYATVIFPGGNKGGGTSCGPAQNPPNNQTPSS) or having an amino acid sequence with at least 60, 65, 70, 75, 80, 85, 90, 95, 96, 97, 98 or 99% identity with SEQ ID NO: 8.

In one embodiment, the TREM-1 inhibitor is a TLT-1 peptide comprising or having an amino acid sequence selected from the group comprising or consisting of SEQ ID NO: 9 (LQEEDAGEYGCMVDGAR) also referred to as LR17, SEQ ID NO: 10 (LQEEDAGEYGCM) also referred to as LR12, SEQ ID NO: 11 (LQEEDA) also referred to as LR6-1, SEQ ID NO: 12 (EDAGEY) also referred to as LR6-2, SEQ ID NO: 13 (GEYGCM) also referred to as LR6-3, and sequences having at least 60, 65, 70, 75, 80, 85, 90, 95, 96, 97, 98 or 99% identity with SEQ ID NO: 9, SEQ ID NO: 10, SEQ ID NO: 11, SEQ ID NO: 12, or SEQ ID NO: 13.

In one embodiment, the TREM-1 inhibitor is a TLT-1 peptide consisting of 6 to 12, 13, 14, 15, 16, 17, 18, 19, or to 20 amino acids and comprising an amino acid sequence selected from the group comprising or consisting of SEQ ID NO: 9, SEQ ID NO: 10, SEQ ID NO: 11, SEQ ID NO: 12, SEQ ID NO: 13, and sequences having at least 60, 65, 70, 75, 80, 85, 90, 95, 96, 97, 98 or 99% identity with SEQ ID NO: 9, SEQ ID NO: 10, SEQ ID NO: 11, SEQ ID NO: 12, or SEQ ID NO: 13.

In one embodiment, the TREM-1 inhibitor is a TLT-1 peptide consisting of 6 to 12, 13, 14, 15, 16, or to 17 amino acids and comprising or consisting of at least 6 consecutive amino acids from the amino acid sequence as set forth in SEQ ID NO: 9 or from an amino acid sequence with at least 60, 65, 70, 75, 80, 85, 90, 95, 96, 97, 98 or 99% identity with SEQ ID NO: 9.

In one embodiment, the TREM-1 inhibitor is a TLT-1 peptide consisting of 12 to 13, 14, 15, 16, 17, 18, 19, or to 20 amino acids and comprising an amino acid sequence as set forth in SEQ ID NO: 10 or an amino acid sequence with at least 60, 65, 70, 75, 80, 85, 90, 95, 96, 97, 98 or 99% identity with SEQ ID NO: 10.

In one embodiment, the TREM-1 inhibitor is a TLT-1 peptide consisting of an amino acid sequence selected from the group comprising or consisting of SEQ ID NO: 9, SEQ ID NO: 10, SEQ ID NO: 11, SEQ ID NO: 12, SEQ ID NO: 13, and sequences having at least 60, 65, 70, 75, 80, 85, 90, 95, 96, 97, 98 or 99% identity with SEQ ID NO: 9, SEQ ID NO: 10, SEQ ID NO: 11, SEQ ID NO: 12, or SEQ ID NO: 13.

In one embodiment, the TREM-1 inhibitor is a TLT-1 peptide having an amino acid sequence as set forth in SEQ ID NO: 10, also known as LR12 or nangibotide or motrem, or a sequence having at least 60, 65, 70, 75, 80, 85, 90, 95, 96, 97, 98 or 99% identity with SEQ ID NO: 10.

In one embodiment, the TLT-1 peptide as described hereinabove has a D- or L-configuration.

In one embodiment, the amino acid from the amino end of the TLT-1 peptide as described hereinabove has an acetylated terminal amino group, and the amino acid from the carboxyl end has an amidated terminal carboxy group.

In one embodiment, the TLT-1 peptide as described hereinabove may undergo reversible chemical modifications in order to increase its bioavailability (including stability and fat solubility) and its ability to pass the blood-brain barrier and epithelial tissue. Examples of such reversible chemical modifications include esterification of the carboxy groups of glutamic and aspartic amino acids with an alcohol, thereby removing the negative charge of the amino acid and increasing its hydrophobicity. This esterification is reversible, as the ester link formed is recognized by intracellular esterases which hydrolyze it, restoring the charge to the aspartic and glutamic residues. The net effect is an accumulation of intracellular peptide, as the internalized, de-esterified peptide cannot cross the cell membrane.

Another example of such reversible chemical modifications includes the addition of a further peptide sequence, which allows the increase of the membrane permeability, such as a TAT peptide or Penetratin peptide (see—Charge-Dependent Translocation of the Trojan. A Molecular View on the Interaction of the Trojan Peptide Penetratin with the 15 Polar Interface of Lipid Bilayers. Biophysical Journal, Volume 87, Issue 1, 1 Jul. 2004, Pages 332-343).

The TLT-1 peptide as described hereinabove may be obtained through conventional methods of solid-phase chemical peptide synthesis, following Fmoc and/or Boc-based methodology (see Pennington, M. W. and Dunn, B. N. (1994). Peptide synthesis protocols. Humana Press, Totowa.). Alternatively, the TLT-1 peptide as described hereinabove may be obtained through conventional methods based on recombinant DNA technology, e.g., through a method that, in brief, includes inserting the nucleic acid sequence coding for the peptide into an appropriate plasmid or vector, transforming competent cells for said plasmid or vector or transfecting mammal cells, and growing said cells under conditions that allow the expression of the peptide and, if desired, isolating and (optionally) purifying the peptide through conventional means known to experts in these matters. A review of the principles of recombinant DNA technology may be found, for example, in the text book entitled “Principles of Gene Manipulation: An Introduction to Genetic Engineering,” R. W. Old & S. B. Primrose, published by Blackwell Scientific Publications, 4th Edition (1989).

In one embodiment, the TREM-1 inhibitor is an antibody directed to (or against) TREM-1, sTREM-1, TREM-1 ligand and/or sTREM-1 ligand. In other words, in one embodiment, the TREM-1 inhibitor is an antibody recognizing TREM-1, sTREM-1, TREM-1 ligand and/or sTREM-1 ligand; that is to say an antibody specific for TREM-1, soluble TREM-1 (sTREM-1), TREM-1 ligand and/or sTREM-1 ligand.

In one embodiment, the antibody of the invention is a monoclonal antibody.

In one embodiment, the TREM-1 inhibitor is an antibody, or an antigen-binding thereof, directed to (or against) TREM-1, i.e., a TREM-1 antibody (also referred to as an anti-TREM-1 antibody).

According to one embodiment, the TREM-1 inhibitor, preferably the TLT-1 peptide as described hereinabove, is for administration by infusion, preferably by intravenous infusion. In one embodiment, the TREM-1 inhibitor, preferably the TLT-1 peptide as described hereinabove, is for administration by continuous infusion, preferably by continuous intravenous infusion.

According to one embodiment, the TREM-1 inhibitor, preferably the TLT-1 peptide as described hereinabove, is for administration at a dose ranging from about 0.1 mg/kg/h to about 3 mg/kg/h (mg per kg bodyweight per hour), preferably from about 0.3 mg/kg/h to about 1 mg/kg/h.

In one embodiment, the TREM-1 inhibitor, preferably the TLT-1 peptide as described hereinabove, is for administration at a dose ranging from about 0.1 mg/kg/h to about 1 mg/kg/h.

In one embodiment, the TREM-1 inhibitor, preferably the TLT-1 peptide as described hereinabove, is for administration at a dose of about 0.1, 0.2, 0.3, 0.4, 0.5, 0.6, 0.7, 0.8, 0.9 or 1 mg/kg/h.

According to one embodiment, the TREM-1 inhibitor, preferably the TLT-1 peptide as described hereinabove, is for administration at a dose ranging from about 0.170 g/24 h to about 4.5 g/24 h, preferably from about 0.5 g/24 h to about 1.7 g/24. In other words, the TREM-1 inhibitor, preferably the TLT-1 peptide as described hereinabove, is for administration at a daily dose ranging from about 0.170 g to about 4.5 g, preferably at a daily dose ranging from about 0.5 g to about 1.7 g.

In one embodiment, the TREM-1 inhibitor, preferably the TLT-1 peptide as described hereinabove, is for administration at a dose ranging from about 0.170 g/24 h to about 1.7 g/24 h. In other words, the TREM-1 inhibitor, preferably the TLT-1 peptide as described hereinabove, is for administration at a daily dose ranging from about 0.170 g to about 1.7 g.

In one embodiment, the TREM-1 inhibitor, preferably the TLT-1 peptide as described hereinabove, is for administration at a daily dose of about 0.17, 0.33, 0.5, 0.67, 0.84, 1, 1.17, 1.34, 1.5 or 1.7 g.

In one embodiment, the TREM-1 inhibitor, preferably the TLT-1 peptide as described hereinabove, is for administration for at least 24 hours.

In one embodiment, the TREM-1 inhibitor, preferably the TLT-1 peptide as described hereinabove, is for administration for 1, 2, 3, 4, or 5 day(s). In one embodiment, the TREM-1 inhibitor, preferably the TLT-1 peptide as described hereinabove, is for administration for at most 5 days.

According to one embodiment, the TREM-1 inhibitor, preferably the TLT-1 peptide as described hereinabove, is for administration with at least one further pharmaceutically active agent.

According to the present invention, the TREM-1 inhibitor, preferably the TLT-1 peptide as described hereinabove, may be administered simultaneously, separately, or sequentially with said at least one further pharmaceutically active agent.

Examples of further pharmaceutically active agents that may be administered to a subject suffering from a disease caused by a coronavirus as described hereinabove include, without being limited to, antiviral agents, anti-interleukin 6 (anti-IL-6) agents, steroids; anti-coagulants; and other agents such as baricitinib, chloroquine or hydroxychloroquine.

In one embodiment, the at least one further pharmaceutically active agent is an antiviral agent, an anti-IL-6 agent, a steroid, an anti-coagulant, baricitinib (in particular baricitinib in combination with remdesivir), chloroquine, hydroxychloroquine, or any mixes thereof. In one embodiment, the at least one further pharmaceutically active agent is an antiviral agent, an anti-IL-6 agent, chloroquine, hydroxychloroquine, or any mixes thereof.

Example of antiviral agents that may be administered to a subject suffering from a disease caused by a coronavirus as described hereinabove include, without being limited to, remdesivir, and a combination of lopinavir and ritonavir (lopinavir/ritonavir).

In one embodiment, the at least one further pharmaceutically active agent is remdesivir, or a combination of lopinavir and ritonavir (lopinavir/ritonavir). In one embodiment, the at least one further pharmaceutically active agent is remdesivir, alone or in combination with baricitinib.

As used herein, anti-IL-6 agents target either IL-6 (interleukin 6 or interleukin-6) or its receptor (IL-6R). Example of anti-IL-6 agents that may be administered to a subject suffering from a disease caused by a coronavirus as described hereinabove include, without being limited to, tocilizumab and sarilumab.

In one embodiment, the at least one further pharmaceutically active agent is an anti-IL-6 agent. In one embodiment, the at least one further pharmaceutically active agent is tocilizumab or sarilumab.

In one embodiment, the at least one further pharmaceutically active agent is a steroid, such as dexamethasone.

In one embodiment, the at least one further pharmaceutically active agent is an anti-coagulant. Example of anti-coagulants that may be administered to a subject suffering from a disease caused by a coronavirus as described hereinabove include, without being limited to, heparin (in particular low-molecular-weight heparin or LMWH), and fondaparinux. In one embodiment, the at least one further pharmaceutically active agent is heparin (in particular low-molecular-weight heparin or LMWH) or fondaparinux.

In one embodiment, the at least one further pharmaceutically active agent is baricitinib, alone or in combination with remdesivir.

In one embodiment, the at least one further pharmaceutically active agent is selected from the group comprising or consisting of remdesivir, a combination of lopinavir and ritonavir, tocilizumab, sarilumab, chloroquine, hydroxychloroquine, and any mixes thereof. In one embodiment, the at least one further pharmaceutically active agent is selected from the group comprising or consisting of remdesivir (alone or in combination with baricitinib), a combination of lopinavir and ritonavir, tocilizumab, sarilumab, dexamethasone, an anti-coagulant such as heparin (in particular low-molecular-weight heparin or LMWH) or fondaparinux, and any mixes thereof.

Another object of the present invention is a method for treating a disease caused by a coronavirus, preferably COVID-19 caused by SARS-CoV-2, in a subject in need thereof, said method comprising administering to the subject a TREM-1 inhibitor, preferably a TLT-1 peptide, as described hereinabove.

In one embodiment, the method of the invention comprises administering a therapeutically effective amount of a TREM-1 inhibitor, preferably a TLT-1 peptide, as described hereinabove.

In one embodiment, the method of the invention comprises administering at least one further pharmaceutically active agent as described hereinabove.

Another object of the present invention is a pharmaceutical composition for treating or for use in the treatment of a disease caused by a coronavirus, preferably COVID-19 caused by SARS-CoV-2, in a subject in need thereof, said pharmaceutical composition comprising a TREM-1 inhibitor, preferably a TLT-1 peptide, as described hereinabove and at least one pharmaceutically acceptable excipient.

Another object of the present invention is the use of a TREM-1 inhibitor, preferably a TLT-1 peptide, as described hereinabove, for the manufacture of a medicament for the treatment of a disease caused by a coronavirus, preferably COVID-19 caused by SARS-CoV-2, in a subject in need thereof.

The present invention also relates to a triggering receptor expressed on myeloid cells-1 (TREM-1) level, in particular to a soluble triggering receptor expressed on myeloid cells-1 (sTREM-1) level, as a marker for use in a method for identifying a subject suffering from a disease caused by a coronavirus infection, in particular COVID-19 caused by SARS-CoV-2, susceptible to respond to a therapy, in particular a TREM-1 inhibitor, and in a method for monitoring the effectiveness of a therapy, in particular a TREM-1 inhibitor, administered to a subject suffering from a disease caused by a coronavirus infection, COVID-19 caused by SARS-CoV-2.

The present invention thus relates to methods comprising:
measuring the level of TREM-1, in particular of sTREM-1, in a biological sample from the subject as described herein; and
comparing the level of TREM-1, in particular of sTREM-1, measured in the biological sample from the subject to a reference value as described herein.

As detailed above, TREM-1 is a glycoprotein receptor belonging to the Ig superfamily that is expressed notably on myeloid cells. sTREM-1 is a soluble form of TREM-1 lacking the transmembrane and intracellular domains of TREM-1. Without wishing to be bound to a theory, the Applicant suggests that PRRs (Pathogen Recognition Receptors) engagement, including Nod-like receptors (NLRs) and Toll-like receptors (TLRs), induce the upregulation of TREM-1 expression and/or its mobilization and clustering at the cell membrane, which lead to its dimerization and multimerization. Said NLRs and TLRs activation can occur by linking DAMPs (Danger Associated Molecular Patterns) or PAMPs (Pathogen Associated Molecular Patterns). In particular, said NLRs and TLRs activation can occur under sterile inflammatory conditions by linking DAMPs (Danger Associated Molecular Patterns) and/or alarmins, or under infectious conditions by linking PAMPs (Pathogen Associated Molecular Patterns). This activation of NLRs and TLRs induces the upregulation of proteases, in particular of metalloproteinases, which in turn, among a number of targets, will induce the liberation of a soluble TREM-1 through proteolytic cleavage of membrane-anchored TREM-1 (Gomez-Pina et al., J Immunol. 2007 Sep. 15; 179(6):4065-73). Said proteolytic cleavage depends on the dimerization of the TREM-1 receptor. sTREM-1 is thus shed from the membrane of myeloid cells, in particular from activated myeloid cells and sTREM-1 release is a marker of TREM-1 activation. In one embodiment, sTREM-1 corresponds to the soluble form of the extracellular domain of TREM-1. In one embodiment, sTREM-1 corresponds to a truncated TREM-1 shed from the membrane of myeloid cells, in particular from activated myeloid cells.

According to one embodiment, the level of TREM-1 is the level of TREM-1 transcript.

Example of known TREM-1 transcripts include, without being limited to, the transcript commonly referred to as TREM1-201 (transcript ID ensembl ENST00000244709.8) corresponding to SEQ ID NO: 1; the transcript commonly referred to as TREM1-202, also known as TREM-1 isoform 2 (ensembl transcript ID ENST00000334475.10) corresponding to SEQ ID NO: 2; the transcript commonly referred to as TREM1-207, also known as TREM-1 isoform 3 (ensembl transcript ID ENST00000591620.1) corresponding to SEQ ID NO: 3, the transcript commonly referred to as TREM1-204 (ensembl transcript ID ENST00000589614.5) corresponding to SEQ ID NO: 4.

In one embodiment, the TREM-1 transcript has an amino acid sequence as set forth in SEQ ID NO: 1, SEQ ID NO: 2, SEQ ID NO: 3, or SEQ ID NO: 4.

In one embodiment, the TREM-1 transcript has an amino acid sequence as set forth in SEQ ID NO: 1, SEQ ID NO: 3, or SEQ ID NO: 4.

In one embodiment, sTREM-1 is a variant of SEQ ID NO: 1, a variant of SEQ ID NO: 3, or a variant of SEQ ID NO: 4.

In one embodiment, a variant of SEQ ID NO: 1, SEQ ID NO: 3, or SEQ ID NO: 4 is an amino acid sequence comprising or consisting of at least 25 contiguous amino acids, preferably at least 50, 60, 70, 80, 90, 100, 110, 120, 130, 140, 150, 160, 165, 170, 175, 180, or 185 contiguous amino acids of the amino acid sequence of SEQ ID NO: 1, SEQ ID NO: 3, or SEQ ID NO: 4, respectively.

In one embodiment, a variant of SEQ ID NO: 1, SEQ ID NO: 3, or SEQ ID NO: 4 is an amino acid sequence comprising the amino acid sequence of SEQ ID NO: 1, SEQ ID NO: 3, or SEQ ID NO: 4, respectively, and additional amino acids at the C-terminus and/or at the N-terminus of SEQ ID NO: 1, SEQ ID NO: 3, or SEQ ID NO: 4, respectively, wherein the number of additional amino acids ranges from 1 to 50, preferably from 1 to 20, more preferably from 1 to 10 amino acids, such as, for example, 1, 2, 3, 4, 5, 6, 7, 8, 9, or 10 amino acids at the C-terminus and/or 1, 2, 3, 4, 5, 6, 7, 8, 9, or 10 amino acids at the N-terminus.

In one embodiment, a variant of SEQ ID NO: 1, SEQ ID NO: 3, or SEQ ID NO: 4 is an amino acid sequence that typically differs from the amino acid sequence of SEQ ID NO: 1, SEQ ID NO: 3, or SEQ ID NO: 4, respectively, through one or more amino acid substitution(s), deletion(s), addition(s) and/or insertion(s). In one embodiment, said substitution(s), deletion(s), addition(s) and/or insertion(s) may affect 1, 2, 3, 4, 5, 6, 7, 8, 9, or 10 amino acids.

In one embodiment, a variant of SEQ ID NO: 1, SEQ ID NO: 3, or SEQ ID NO: 4 is an amino acid sequence of at least 25 amino acids, preferably of at least 50, 60, 70, 80, 90, 100, 110, 120, 130, 140, 150, 160, 165, 170, 175, 180, or 185 amino acids having at least 60%, 65%, 70%, 75%, 80%, 90%, 95%, or at least 96%, 97%, 98%, 99% or more identity with the amino acid sequence of SEQ ID NO: 1, SEQ ID NO: 3, or SEQ ID NO: 4.

In one embodiment, a variant of SEQ ID NO: 1, SEQ ID NO: 3, or SEQ ID NO: 4 is an amino acid sequence having at least 60%, 65%, 70%, 75%, 80%, 90%, 95%, or at least 96%, 97%, 98%, 99% or more identity with the amino acid sequence of SEQ ID NO: 1, SEQ ID NO: 3, or SEQ ID NO: 4.

According to one embodiment, the level of TREM-1 is the level of sTREM-1.

In one embodiment, sTREM-1 corresponds to the extracellular fragment generated by cleavage of the membrane-bound TREM-1 having an amino acid sequence as set forth in SEQ ID NO: 1 by a protease, preferably a matrix metallopeptidase, more preferably by the matrix metalloproteinase 9 (MMP9).

In one embodiment, sTREM-1 has an amino acid sequence as set forth in SEQ ID NO: 5 (ATKLTEEKYELKEGQTLDVKCDYTLEKFASSQKAWQIIRDG EMPKTLACTERPSKNSHPVQVGRIILEDYHDHGLLRVRMVNLQVEDSGLYQCV IYQPPKEPHMLFDRIRLVVTKGFSGTPGSNENSTQNVYKIPPTTTKALCPLYTSP RTVTQAPPKSTADVSTPDSEINLTNVTDIIRVPVFN), corresponding to amino acids 21 to 205 of SEQ ID NO: 1. In one embodiment, sTREM-1 has an amino acid sequence as set forth in SEQ ID NO: 6 (LKEGQTLDVKCDYTLEKFASSQKAW QIIRDGEMPKTLACTERPSKNSHPVQVGRIILEDYHDHGLLRVRMVNLQVEDSG LYQCVIYQPPKEPHMLFDRIRLVVTKGFSGTPGSNENSTQNVYKIPPTTTKALCP LYTSPRTVTQAPPKSTADVSTPDSEINLTNVTDIIRVPVFN), corresponding to amino acids 31 to 205 of SEQ ID NO: 1.

sTREM-1 may also result from an alternative splicing of TREM-1 mRNA. A TREM-1 splice variant was characterized in 2015 by Baruah et al., (J Immunol. 2015 Dec. 15; 195(12):5725-31) and was found to be secreted from primary and secondary human neutrophil granules. In one embodiment, sTREM-1 corresponds to a TREM-1 splice variant. In one embodiment, sTREM-1 corresponds to the TREM-1 transcript commonly referred to as TREM1-202, also known as TREM-1 isoform 2, encoding an amino acid sequence as set forth in SEQ ID NO: 2. In one embodiment, sTREM-1 thus has an amino acid sequence as set forth in SEQ ID NO: 2 (MRKTRLWGLLWMLFVSELRAATKLTEEKYELKEGQTLDVKCDYTLEKFASSQ KAWQIIRDGEMPKTLACTERPSKNSHPVQVGRIILEDYHDHGLLRVRMVNLQV EDSGLYQCVIYQPPKEPHMLFDRIRLVVTKGFRCSTLSFSWLVDS).

In one embodiment, sTREM-1 comprises an amino sequence as set forth in SEQ ID NO: 7 (LKEGQTLDVKCDYTLEKFASSQKAWQIIRDGEMPKTLAC TERPSKNSHPVQVGRIILEDYHDHGLLRVRMVNLQVEDSGLYQCVIYQPPKEPH MLFDRIRLVVTKGF), corresponding to amino acids 31 to 137 of SEQ ID NO: 1, and has a length of 200 amino acids or less, preferably of 185 amino acids or less.

In one embodiment, sTREM-1 has an amino acid sequence as set forth in SEQ ID NO: 2, SEQ ID NO: 5, or SEQ ID NO: 6. In one embodiment, sTREM-1 has an amino acid sequence as set forth in SEQ ID NO: 5 or in SEQ ID NO: 6.

In one embodiment, sTREM-1 is a variant of SEQ ID NO: 2, a variant of SEQ ID NO: 5, or a variant of SEQ ID NO: 6. In one embodiment, sTREM-1 is a variant of SEQ ID NO: 5 or a variant of SEQ ID NO: 6.

In one embodiment, a variant of SEQ ID NO: 2, SEQ ID NO: 5, or SEQ ID NO: 6 is an amino acid sequence comprising or consisting of at least 25 contiguous amino acids, preferably at least 50, 60, 70, 80, 90, 100, 110, 120, 130, 140, 150, 160, 165, 170, 175, 180, or 185 contiguous amino acids of the amino acid sequence of SEQ ID NO: 2, SEQ ID NO: 5, or SEQ ID NO: 6, respectively.

In one embodiment, a variant of SEQ ID NO: 2, SEQ ID NO: 5, or SEQ ID NO: 6 is an amino acid sequence comprising the amino acid sequence of SEQ ID NO: 2, SEQ ID NO: 5, or SEQ ID NO: 6, respectively, and additional amino acids at the C-terminus and/or at the N-terminus of SEQ ID NO: 2, SEQ ID NO: 5, or SEQ ID NO: 6, respectively, wherein the number of additional amino acids ranges from 1 to 50, preferably from 1 to 20, more preferably from 1 to 10 amino acids, such as, for example, 1, 2, 3, 4, 5, 6, 7, 8, 9, or 10 amino acids at the C-terminus and/or 1, 2, 3, 4, 5, 6, 7, 8, 9, or 10 amino acids at the N-terminus.

In one embodiment, a variant of SEQ ID NO: 2, SEQ ID NO: 5, or SEQ ID NO: 6 is an amino acid sequence that typically differs from the amino acid sequence of SEQ ID NO: 2, SEQ ID NO: 5, or SEQ ID NO: 6, respectively, through one or more amino acid substitution(s), deletion(s), addition(s) and/or insertion(s). In one embodiment, said substitution(s), deletion(s), addition(s) and/or insertion(s) may affect 1, 2, 3, 4, 5, 6, 7, 8, 9, or 10 amino acids.

In one embodiment, a variant of SEQ ID NO: 2, SEQ ID NO: 5, or SEQ ID NO: 6 is an amino acid sequence of at least 25 amino acids, preferably of at least 50, 60, 70, 80, 90, 100, 110, 120, 130, 140, 150, 160, 165, 170, 175, 180, or 185 amino acids having at least 60%, 65%, 70%, 75%, 80%, 90%, 95%, or at least 96%, 97%, 98%, 99% or more identity with the amino acid sequence of SEQ ID NO: 2, SEQ ID NO: 5, or SEQ ID NO: 6, respectively.

In one embodiment, a variant of SEQ ID NO: 2, SEQ ID NO: 5, or SEQ ID NO: 6 is an amino acid sequence having at least 60%, 65%, 70%, 75%, 80%, 90%, 95%, or at least 96%, 97%, 98%, 99% or more identity with the amino acid sequence of SEQ ID NO: 2, SEQ ID NO: 5, or SEQ ID NO: 6, respectively.

In one embodiment, the variant of SEQ ID NO: 2, SEQ ID NO: 5, or SEQ ID NO: 6 is not SEQ ID NO: 1.

In one embodiment, sTREM-1 is a fragment of SEQ ID NO: 2, a fragment of SEQ ID NO: 5, or a fragment of SEQ ID NO: 6. In one embodiment, sTREM-1 is a fragment of SEQ ID NO: 5 or a fragment of SEQ ID NO: 6.

In one embodiment, a fragment of SEQ ID NO: 2, SEQ ID NO: 5, or SEQ ID NO: 6 is an amino acid sequence comprising or consisting of at least 25 contiguous amino acids, preferably of at least 50, 60, 70, 80, 90, 100, 110, 120, 130, 140, 150, 160, 165, 170, 175, 180, or 185 contiguous amino acids of the amino acid sequence of SEQ ID NO: 2, SEQ ID NO: 5, or SEQ ID NO: 6, respectively. In one embodiment, a fragment of SEQ ID NO: 2 is an amino acid sequence comprising or consisting of at least 25 contiguous amino acids, preferably of at least 50, 60, 70, 80, 90, 100, 110, 120, 130, 135, 140, or 145 contiguous amino acids of the amino acid sequence of SEQ ID NO: 2. In one embodiment, a fragment of SEQ ID NO: 5 is an amino acid sequence comprising or consisting of at least 25 contiguous amino acids, preferably of at least 50, 60, 70, 80, 90, 100, 110, 120, 130, 140, 150, 160, 165, 170, 175, or 180 contiguous amino acids of the amino acid sequence of SEQ ID NO: 5. In one embodiment, a fragment of SEQ ID NO: 6 is an amino acid sequence comprising or consisting of at least 25 contiguous amino acids, preferably of at least 50, 60, 70, 80, 90, 100, 110, 120, 130, 140, 150, 155, 160, 165 or 170 contiguous amino acids of the amino acid sequence of SEQ ID NO: 6.

According to the methods as described herein, the level of TREM-1, in particular the level of sTREM-1, is measured in a biological sample from a subject as described hereinabove.

As used herein, “biological sample” refers to a biological sample isolated, collected or harvested from a subject and can include, by way of example and not limitation, bodily fluids, cell samples and/or tissue extracts such as homogenates or solubilized tissues obtained from a subject.

In one embodiment, the present invention does not comprise obtaining a biological sample from a subject. Thus, in one embodiment, the biological sample from the subject is a biological sample previously obtained from the subject. Said biological sample may be conserved in adequate conditions before being used as described herein.

In one embodiment, the biological sample from the subject is a body fluid sample. Examples of body fluids include, without being limited to, blood, plasma, serum, lymph, urine, bronchioalveolar lavage fluid, cerebrospinal fluid, sweat or any other bodily secretion or derivative thereof.

According to the present invention, “blood” includes whole blood, plasma, serum, circulating epithelial cells, constituents, or any other derivative of blood.

In one embodiment, the biological sample from the subject is a blood sample. In one embodiment, the biological sample from the subject is a whole blood sample or a plasma sample. Methods for obtaining a plasma sample are routinely used in clinical laboratories. In one embodiment, the whole blood sample or the plasma sample from the subject is processed to obtain a serum sample. Methods for obtaining a serum sample from a whole blood sample or a plasma sample are routinely used in clinical laboratories.

In one embodiment, the biological sample is a blood sample, a plasma sample or a serum sample. Accordingly, in one embodiment, the TREM-1 level, in particular the sTREM-1 level, is a blood level, a plasma level or a serum level.

In one embodiment, the biological sample from the subject is a tissue extract. Tissue extracts are obtained routinely from tissue biopsy and autopsy material.

As used herein, the term “level” refers to the expression level of TREM-1, in particular of sTREM-1. It can refer alternatively to the transcription level of TREM-1, in particular of sTREM-1 (i.e., the level of mRNA or cDNA) or to the translation level of TREM-1, in particular of sTREM-1 (i.e., the level of protein). The expression level may be detected intracellularly or extracellularly.

According to the present invention, the level of TREM-1, in particular of sTREM-1, may be measured by any known method in the art. Methods for measuring an expression level such as a transcription level or a translation level are well-known to the skilled artisan.

In one embodiment, the term “level” refers to the quantity, amount or concentration of TREM-1, in particular of sTREM-1. Thus, the level of TREM-1, in particular of sTREM-1, measured in a biological sample from a subject refers to the quantity, amount or concentration of TREM-1, in particular of sTREM-1, in said biological sample.

According to one embodiment, the level of TREM-1, in particular of sTREM-1, refers to a protein level, a protein quantity, a protein amount or a protein concentration.

In one embodiment, the level of TREM-1 refers to the level of an amino acid sequence as set forth in SEQ ID NO: 1, SEQ ID NO: 3 and/or SEQ ID NO: 4, and/or variants thereof as described hereinabove.

In one embodiment, the level of TREM-1 is a level of sTREM-1.

In one embodiment, the level of sTREM-1 refers to the level of an amino acid sequence as set forth in SEQ ID NO: 2, SEQ ID NO: 5 and/or SEQ ID NO: 6, and/or fragments and/or variants thereof as described hereinabove. In one embodiment, the level of sTREM-1 refers to the level of an amino acid sequence as set forth in SEQ ID NO: 5 and/or SEQ ID NO: 6, and/or fragments and/or variants thereof as described hereinabove.

Methods for measuring the translation level of TREM-1, in particular of sTREM-1 (i.e., the level TREM-1 protein or of sTREM-1 protein) are well-known to the skilled artisan and include, without being limited to, immunohistochemistry, multiplex methods (such as Luminex®), immunoassays, western blot, enzyme-linked immunosorbent assay (ELISA), sandwich ELISA, multiplex ELISA, capillary-based ELISA (such as the ELLA® platform), electrochemiluminescence (ECL) also referred as electrochemiluminescence immunoassay (ECLIA), enzyme-linked fluorescent assay (ELFA), fluorescent-linked immunosorbent assay (FLISA), enzyme immunoassay (EIA), radioimmunoassay (RIA), flow cytometry (FACS), surface plasmon resonance (SPR), biolayer interferometry (BLI), immunochromatographic assay (ICA) (such as NEXUS IB10, Sphingotech) and mass spectrometry-based approaches.

Typically, measuring the level of TREM-1 protein, in particular of sTREM-1 protein, in a biological sample as described hereinabove may comprise contacting the biological sample with a binding partner capable of selectively interacting with sTREM-1 in the biological sample.

In one embodiment, measuring the level of TREM-1 protein, in particular of sTREM-1 protein, in a biological sample as described hereinabove comprises the use of an antibody, such as a polyclonal or a monoclonal antibody.

Examples of antibodies allowing the detection of TREM-1, in particular of sTREM-1, include, without being limited to, the polyclonal antibody raised against Met1-Arg200 amino acids of human TREM-1 (reference AF1278 from R&D Systems), the monoclonal antibody raised against Ala21-Asn205 of human TREM-1 (reference MAB1278 from R&D Systems), the purified anti-human CD354 (TREM-1) antibody (clone TREM-26, reference 314902 from BioLegend), the purified anti-human CD354 (TREM-1) antibody (clone TREM-37, reference 316102 from BioLegend), the monoclonal mouse anti-human sTREM1 (clone 15G7, reference 298099 from USBio), the mouse anti-human TREM1 (clone 2E2, reference 134704 from USBio). Other non-limitative examples of antibodies allowing the detection of sTREM-1 include sTREM-1 and/or TREM-1 antibodies described in the following patents or patent applications: US2013/150559, US 2013/211050, US 2013/309239, WO2013/120553 and U.S. Pat. No. 8,106,165.

In one embodiment, measuring the level of TREM-1, in particular of sTREM-1, in particular the level of sTREM-1 protein, in a biological sample as described hereinabove comprises the use of an ELISA, an ECLIA or an ELFA.

An ELISA may thus be used for measuring the level of TREM-1, in particular of sTREM-1, in a biological sample, wherein for example the wells of an assay plate are coated with at least one antibody which recognizes TREM-1, or sTREM-1. A biological sample containing or suspected of containing TREM-1, or sTREM-1, is then added to the coated wells. After a period of incubation sufficient to allow the formation of antibody-TREM-1 complexes, or antibody-sTREM-1 complexes, the plate can be washed to remove unbound moieties and a detectably labelled secondary binding molecule added, such as, for example, a second antibody which recognizes TREM-1, or sTREM-1, coupled to horseradish peroxidase (HRP). The secondary binding molecule is allowed to react with any captured antibody-TREM-1 complexes, or antibody-sTREM-1 complexes, the plate washed and the presence of the secondary binding molecule detected using methods well-known in the art. It is understood that commercial assay enzyme-linked immunosorbent assay (ELISA) kits are available. Examples of ELISAs thus include, without being limited to, the TREM-1 Quantikine ELISA kit (reference DTRM10C from R&D Systems); the human TREM-1 DuoSet (references DY1278B and DY1278BE from R&D Systems), the sTREM-1 ELISA (reference sTREM-1 ELISA from iQProducts). Examples of ELISAs include, without being limited to, the TREM-1 Quantikine ELISA kit (reference DTRM10C from R&D Systems); the human TREM-1 DuoSet (references DY1278B and DY1278BE from R&D Systems), the sTREM-1 ELISA (reference sTREM-1 ELISA from iQProducts).

An ECLIA may also be used for measuring the level of TREM-1, in particular of sTREM-1, in a biological sample, wherein for example a biological sample containing or suspected of containing TREM-1, or sTREM-1, is incubated with at least two antibodies which recognize TREM-1, or sTREM-1, on different epitopes in order to form sandwich antibodies-TREM-1 or antibodies-sTREM-1 complexes, with one of the antibody being biotinylated (i.e., the capture antibody) and the other being labeled with a ruthenium complex (i.e., the detection antibody). After a period of incubation sufficient to allow the formation of the sandwich complexes, streptavidin-coated microparticles (such as streptavidin-coated magnetic beads) are added so that the sandwich complexes become bound to the particles via interaction of biotin and streptavidin. Once in the measuring cell of an analyzer, the microparticles are magnetically captured onto the surface of an electrode. Unbound moieties are removed and a voltage is applied to the electrode, thus exciting the ruthenium complex which then emits light at 620 nm. The light emitted is measured by a photomultiplier in the measuring cell of the analyzer. Examples of ECLIAs include Elecsys® (Roche Diagnostics).

An ELFA may also be used for measuring the level of TREM-1, in particular of sTREM-1, in a biological sample, wherein for example a receptacle is coated with at least one antibody which recognizes TREM-1, or sTREM-1. A biological sample containing or suspected of containing TREM-1, or sTREM-1, is then added to the receptacle. After a period of incubation sufficient to allow the formation of antibody-TREM-1 complexes, or antibody-sTREM-1 complexes, the receptacle can be washed to remove unbound moieties and a secondary binding molecule labeled with an enzyme (such as alkaline phosphatase) is added. The secondary binding molecule is allowed to react with any captured antibody-TREM-1 complexes, or antibody-sTREM-1 complexes, the receptacle washed and the presence of the secondary binding molecule detected through the measurement of the fluorescence emitted upon addition of a substrate of the enzyme (such as 4-methyl-umbelliferyl phosphate), which becomes fluorescent after hydrolysis by the enzyme. Examples of ELFAs include VIDAS® (Biomérieux).

According to one embodiment, the level of TREM-1, in particular sTREM-1, refers to a nucleic acid level, a nucleic acid quantity, a nucleic acid amount or a nucleic acid concentration. In one embodiment, the nucleic acid is a RNA, preferably a mRNA, or a cDNA.

In one embodiment, the level of TREM-1 is a level of TREM-1 transcript.

In one embodiment, the level of TREM-1 nucleic acid refers to the level of mRNA or cDNA encoding an amino acid sequence as set forth in SEQ ID NO: 1, SEQ ID NO: 3 and/or SEQ ID NO: 4, and/or variants thereof as described hereinabove.

In one embodiment, the level of TREM-1 is a level of sTREM-1.

In one embodiment, the level of sTREM-1 nucleic acid refers to the level of mRNA or cDNA encoding an amino acid sequence as set forth in SEQ ID NO: 2, SEQ ID NO: 5 and/or SEQ ID NO: 6, and/or fragments and/or variants thereof as described hereinabove. In one embodiment, the level of sTREM-1 nucleic acid refers to the level of mRNA or cDNA encoding an amino acid sequence as set forth in SEQ ID NO: 5 and/or SEQ ID NO: 6, and/or fragments and/or variants thereof as described hereinabove.

Methods for measuring the transcription level of TREM-1, in particular of sTREM-1, (i.e., the level of TREM-1 mRNA or cDNA, in particular of sTREM-1 mRNA or cDNA) in a biological sample as described hereinabove are well-known to the skilled artisan and include, without being limited to, PCR, qPCR, RT-PCR, RT-qPCR, northern blot, hybridization techniques such as, for example, use of microarrays, and combination thereof including but not limited to, hybridization of amplicons obtained by RT-PCR, sequencing such as, for example, next-generation DNA sequencing (NGS) or RNA-seq (also known as “Whole Transcriptome Shotgun Sequencing”).

In one embodiment, the TREM-1 nucleic acid level, in particular the sTREM-1 nucleic acid level, is measured using the forward and reverse primers having a nucleotide sequence has set forth in SEQ ID NO: 14 and SEQ ID NO: 15, respectively. In one embodiment, the TREM-1 nucleic acid level, in particular the sTREM-1 nucleic acid level, is measured using the forward and reverse primers having a nucleotide sequence has set forth in SEQ ID NO: 16 and SEQ ID NO: 17, respectively. In one embodiment, the TREM-1 nucleic acid level, in particular the sTREM-1 nucleic acid level, is measured using the forward and reverse primers having a nucleotide sequence has set forth in SEQ ID NO: 18 and SEQ ID NO: 19, respectively.

In one embodiment, the level of TREM-1, in particular of sTREM-1, measured in a biological sample as described hereinabove is the level at baseline, i.e., a baseline TREM-1 level, in particular a baseline sTREM-1 level. In one embodiment, a baseline level is the level of TREM-1, in particular of sTREM-1, measured in a biological sample obtained from the subject before the start of medical care, before or at the beginning of the administration of a therapy, upon hospitalization, or upon admission in intensive care unit (ICU).

In one embodiment, the level of TREM-1, in particular of sTREM-1, measured in a biological sample as described hereinabove is the level of TREM-1, in particular of sTREM-1, measured 12 h, 24 h, 36 h, 48 h, 60 h or 72 h following hospitalization (or measured in a biological sample obtained from the subject 12 h, 24 h, 36 h, 48 h, 60 h or 72 h following hospitalization), in particular following admission in intensive care unit (ICU).

In one embodiment, the level of TREM-1, in particular of sTREM-1, measured in a biological sample as described hereinabove is the level of TREM-1, in particular of sTREM-1, measured on day 1, 2, 3, 4, 5, 6, 7, 8, 9, 10, 11, 12, 13, 14, 15, 16, 17, 18, 19, 20, or 21 following hospitalization (or measured in a biological sample obtained from the subject on day 1, 2, 3, 4, 5, 6, 7, 8, 9, 10, 11, 12, 13, 14, 15, 16, 17, 18, 19, 20, or 21 following hospitalization), in particular following admission in intensive care unit (ICU). In other words, in one embodiment, the level of TREM-1, in particular of sTREM-1, measured in a biological sample as described hereinabove is the level of TREM-1, in particular of sTREM-1, measured on the first, second, third, fourth, fifth, sixth or seventh day following hospitalization (or measured in a biological sample obtained from the subject on the first, second, third, fourth, fifth, sixth or seventh day following hospitalization), in particular following admission in intensive care unit (ICU).

In one embodiment, the level of TREM-1, in particular of sTREM-1, measured in a biological sample as described hereinabove is the level of TREM-1, in particular of sTREM-1, measured on day 3 following hospitalization (or measured in a biological sample obtained from the subject on day 3 following hospitalization), in particular following admission in intensive care unit (ICU). In other words, in one embodiment, the level of TREM-1, in particular of sTREM-1, measured in a biological sample as described hereinabove is the level of TREM-1, in particular of sTREM-1, measured on the third day following hospitalization (or measured in a biological sample obtained from the subject on the third day following hospitalization), in particular following admission in intensive care unit (ICU).

According to the methods as described herein, the level of TREM-1, in particular of sTREM-1, measured in a biological sample from a subject as described hereinabove is compared to a reference value.

According to one embodiment, the reference value is a reference TREM-1 level, in particular a reference sTREM-1 level, preferably a blood, plasma or serum level.

According to one embodiment, the reference TREM-1 level, in particular the reference sTREM-1 level, is determined using an enzyme-linked immunosorbent assay (ELISA).

According to one embodiment, the reference TREM-1 level, in particular the reference sTREM-1 level, as determined using a given method or assay encompasses corresponding reference TREM-1 levels, in particular corresponding reference sTREM-1 levels, as determined using another method or assay. Starting from levels obtained with a given method or assay, the skilled artisan will know how to determine corresponding levels obtained with another method or assay. Methods to do so include for example (i) measuring the levels with two different methods or assays in the samples obtained from the subjects of a given reference population, such as a reference population as described herein, thus obtaining two sets of measures for said given reference population; and (ii) determining the correlation between the two sets of measures obtained for the given reference population.

In one embodiment, the reference TREM-1 level, in particular the reference sTREM-1 level, as determined using an ELISA encompasses corresponding reference TREM-1 levels, in particular corresponding reference sTREM-1 levels, as determined using another immunoassay, such as an electrochemiluminescence immunoassay (ECLIA). In one embodiment, the reference TREM-1 level, in particular the reference sTREM-1 level, as determined using an ELISA encompasses the corresponding reference TREM-1 level, in particular the corresponding reference sTREM-1 level, as determined using an ECLIA.

In one embodiment, the reference value is derived from a reference population. In one embodiment, the reference value is derived from population studies, including, for example, subjects having a similar age range, or subjects in the same or similar ethnic group.

According to one embodiment, the reference value is derived from the measure of the TREM-1 level, in particular the sTREM-1 level, in a biological sample obtained from one or more subjects who are substantially healthy. In one embodiment, a “substantially healthy subject” is a subject who has not been diagnosed or identified as having or suffering from a disease caused by a coronavirus, in particular COVID-19. In one embodiment, a “substantially healthy subject” is a subject who has not been diagnosed or identified as having or suffering from a disease caused by a coronavirus, in particular COVID-19, or any other infection. In one embodiment, a “substantially healthy subject” is a subject who has not been diagnosed or identified as having or suffering from a disease caused by a coronavirus, in particular COVID-19, or any other disease inducing a response from the immune system or any other disease inducing activation of the TREM-1 pathway. Thus, in one embodiment, the reference value is a reference TREM-1 level, in particular a reference sTREM-1 level, derived from a reference population of subjects who are substantially healthy.

In one embodiment, the reference value derived from a reference population of subjects who are substantially healthy is a TREM-1 level, in particular a sTREM-1 level, preferably a blood, plasma or serum level, ranging from about 20 pg/mL to about 500 pg/mL, preferably from about 50 pg/mL to about 250 pg/mL, more preferably from about 100 pg/mL to about 200 pg/mL. In one embodiment, the reference value derived from a reference population of subjects who are substantially healthy is a TREM-1 level, in particular a sTREM-1 level, preferably a blood, plasma or serum level, ranging from about 20 pg/mL to about 500 pg/mL, preferably from about 50 pg/mL to about 250 pg/mL, more preferably from about 100 pg/mL to about 200 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value derived from a reference population of subjects who are substantially healthy is a TREM-1 level, in particular a sTREM-1 level, preferably a blood, plasma or serum level, of about 50, 75, 100, 125, 150, 175, 200, 225 or 250 pg/mL. In one embodiment, the reference value derived from a reference population of subjects who are substantially healthy is a TREM-1 level, in particular a sTREM-1 level, preferably a blood, plasma or serum level, of about 50, 75, 100, 125, 150, 175, 200, 225 or 250 pg/mL, as determined using an ELISA, or a corresponding TREM-1 level, in particular a corresponding sTREM-1 level, preferably a blood, plasma or serum level, as determined using another immunoassay, in particular an ECLIA. In one embodiment, the reference value derived from a reference population of subjects who are substantially healthy is a TREM-1 level, in particular a sTREM-1 level, preferably a blood, plasma or serum level, of about 50, 55, 60, 65, 70, 75, 80, 85, 90, 95, 100, 105, 110, 115, 120, 125, 130, 135, 140, 145, or 150 pg/mL. In one embodiment, the reference value derived from a reference population of subjects who are substantially healthy is a TREM-1 level, in particular a sTREM-1 level, preferably a blood, plasma or serum level, of about 50, 55, 60, 65, 70, 75, 80, 85, 90, 95, 100, 105, 110, 115, 120, 125, 130, 135, 140, 145, or 150 pg/mL, as determined using an ELISA, or a corresponding TREM-1 level, in particular a corresponding sTREM-1 level, preferably a blood, plasma or serum level, as determined using another immunoassay, in particular an ECLIA. In one embodiment, the reference value derived from a reference population of subjects who are substantially healthy is a TREM-1 level, in particular a sTREM-1 level, preferably a blood, plasma or serum level, of about 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 pg/mL. In one embodiment, the reference value derived from a reference population of subjects who are substantially healthy is a TREM-1 level, in particular a sTREM-1 level, preferably a blood, plasma or serum level, of about 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 pg/mL, as determined using an ELISA, or a corresponding TREM-1 level, in particular a corresponding sTREM-1 level, preferably a blood, plasma or serum level, as determined using another immunoassay, in particular an ECLIA.

According to one embodiment, the reference value is derived from the measure of the TREM-1 level, in particular of the sTREM-1 level, in a biological sample from one or more subjects diagnosed or identified as suffering, or having suffered, from a disease caused by a coronavirus, in particular from COVID-19 caused by SARS-CoV-2. Thus, in one embodiment, the reference value is a reference TREM-1 level, in particular a reference sTREM-1 level, derived from a reference population of subjects diagnosed or identified as suffering, or having suffered, from a disease caused by a coronavirus, in particular from COVID-19 caused by SARS-CoV-2.

In one embodiment, the reference value can be derived from statistical analyses and/or risk prediction data of a reference population as described hereinabove obtained from mathematical algorithms and computed indices of a disease caused by a coronavirus, in particular COVID-19 caused by SARS-CoV-2.

In one embodiment, the reference value derived from a reference population as described hereinabove is the average TREM-1 level, in particular the average sTREM-1 level, of said reference population. In one embodiment, the reference value derived from a reference population as described hereinabove is the median TREM-1 level, in particular the median sTREM-1 level, of said reference population.

In one embodiment, the reference value derived from a reference population as described hereinabove is a TREM-1 tercile (or tertile), in particular a sTREM-1 tercile (or tertile), i.e., the first TREM-1 tercile, in particular the first sTREM-1 tercile, or the second TREM-1 tercile, in particular the second sTREM-1 tercile, of said reference population.

According to this embodiment of the present invention:
the first tercile (or tertile) corresponds to the TREM-1 value or the sTREM-1 value below which a third of the TREM-1 or sTREM-1 levels measured in the reference population lie and above which two thirds of the TREM-1 or sTREM-1 levels measured in the reference population lie; and
the second tercile (or tertile) corresponds to the TREM-1 value or the sTREM-1 value below which two thirds of the TREM-1 or sTREM-1 levels measured in the reference population lie and above which one third of the TREM-1 or sTREM-1 levels measured in the reference population lie.

In one embodiment, the reference value, preferably the reference value derived from a reference population as described hereinabove, is a TREM-1 level, in particular a sTREM-1 level, preferably a blood, plasma or serum level, ranging from about 20 pg/mL to about 6000 pg/mL, preferably from about 30 pg/mL to about 2000 pg/mL, more preferably from about 50 pg/mL to about 1000 pg/mL.

In one embodiment, the reference value, preferably the reference value derived from a reference population as described hereinabove, is a TREM-1 level, in particular a sTREM-1 level, preferably a blood, plasma or serum level, ranging from about 50 pg/mL to about 800 pg/mL, preferably from about 75 pg/mL to about 600 pg/mL, more preferably from about 100 pg/mL to about 400 pg/mL, even more preferably from about 130 pg/mL to about 400 pg/mL. In one embodiment, the reference value, preferably the reference value derived from a reference population as described hereinabove, is a TREM-1 level, in particular a sTREM-1 level, preferably a blood, plasma or serum level, ranging from about 50 pg/mL to about 800 pg/mL, preferably from about 75 pg/mL to about 600 pg/mL, more preferably from about 100 pg/mL to about 400 pg/mL, even more preferably from about 130 pg/mL to about 400 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value, preferably the reference value derived from a reference population as described hereinabove, is a TREM-1 level, in particular a sTREM-1 level, preferably a blood, plasma or serum level, ranging from about 130 pg/mL to about 600 pg/mL, preferably from about 200 pg/mL to about 500 pg/mL, more preferably from about 250 pg/mL to about 400 pg/mL, even more preferably from about 300 pg/mL to about 375 pg/mL. In one embodiment, the reference value, preferably the reference value derived from a reference population as described hereinabove, is a TREM-1 level, in particular a sTREM-1 level, preferably a blood, plasma or serum level, ranging from about 130 pg/mL to about 600 pg/mL, preferably from about 200 pg/mL to about 500 pg/mL, more preferably from about 250 pg/mL to about 400 pg/mL, even more preferably from about 300 pg/mL to about 375 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value, preferably the reference value derived from a reference population as described hereinabove, is a TREM-1 level, in particular a sTREM-1 level, preferably a blood, plasma or serum level, of about 75, 100, 125, 150, 175, 200, 225, 250, 275, 300, 325, 350, 375 or 400 pg/mL. In one embodiment, the reference value, preferably the reference value derived from a reference population as described hereinabove, is a TREM-1 level, in particular a sTREM-1 level, preferably a blood, plasma or serum level, of about 75, 100, 125, 150, 175, 200, 225, 250, 275, 300, 325, 350, 375 or 400 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value, preferably the reference value derived from a reference population as described hereinabove, is a TREM-1 level, in particular a sTREM-1 level, preferably a blood, plasma or serum level, of about 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, or 430 pg/mL. In one embodiment, the reference value, preferably the reference value derived from a reference population as described hereinabove, is a TREM-1 level, in particular a sTREM-1 level, preferably a blood, plasma or serum level, of about 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, or 430 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value, preferably the reference value derived from a reference population as described hereinabove, is a TREM-1 level, in particular a sTREM-1 level, preferably a blood, plasma or serum level, of about 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 pg/mL. In one embodiment, the reference value, preferably the reference value derived from a reference population as described hereinabove, is a TREM-1 level, in particular a sTREM-1 level, preferably a blood, plasma or serum level, of about 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 pg/mL, as determined using an ELISA, or a corresponding TREM-1 level, in particular a corresponding sTREM-1 level, preferably a blood, plasma or serum level, as determined using another immunoassay, in particular an ECLIA.

According to one embodiment, the reference value is a personalized reference value, i.e., the reference value is a TREM-1 level, in particular a sTREM-1 level, measured in a biological sample obtained from the subject.

In one embodiment, the personalized reference value is a TREM-1 level, in particular a sTREM-1 level, measured in a biological sample obtained from the subject at baseline, i.e., a baseline TREM-1 level, in particular a baseline sTREM-1 level. In one embodiment, the sTREM-1 level is a TREM-1 level, in particular a sTREM-1 level, measured in a biological sample obtained from the subject before the start of medical care. In one embodiment, the baseline level is a TREM-1 level, in particular a sTREM-1 level, measured in a biological sample obtained from the subject upon hospitalization or upon admission in ICU. In one embodiment, the baseline level is a TREM-1 level, in particular a sTREM-1 level, measured in a biological sample obtained from the subject before or at the beginning of the administration of a therapy, in particular before or at the beginning of the administration of a TREM-1 inhibitor as described herein.

Another object of the invention is an in vitro method for identifying a subject suffering from a disease caused by a coronavirus as described hereinabove susceptible to respond to a therapy, said method comprising:
measuring the level of TREM-1, in particular of soluble TREM-1 (sTREM-1), in a biological sample from the subject; and
comparing the level of TREM-1, in particular of sTREM-1, measured in the biological sample from the subject to a reference value.

In one embodiment, the present invention relates to an in vitro method for identifying a subject suffering from COVID-19 susceptible to respond to a therapy, said method comprising:
measuring the level of sTREM-1 in a biological sample from the subject as described hereinabove; and
comparing the level of sTREM-1 measured in the biological sample from the subject to a reference value as described hereinabove.

In one embodiment, the present invention relates to an in vitro method for identifying a subject suffering from a severe form and/or at least one complication of a disease caused by a coronavirus, in particular COVID-19, as described hereinabove, susceptible to respond to a therapy.

In one embodiment, a level of TREM-1, in particular of sTREM-1, measured in the biological sample from the subject higher than the reference value as described hereinabove indicates that the subject as described hereinabove is susceptible to respond to a therapy.

In one embodiment, the reference value is derived from the measure of the TREM-1 level, in particular of sTREM-1 level, in a biological sample obtained from one or more subjects who are substantially healthy as defined hereinabove. In one embodiment, the reference value is a reference sTREM-1 level derived from a reference population of subjects who are substantially healthy.

In one embodiment, said reference value is a sTREM-1 level, preferably a blood, plasma or serum level, ranging from about 20 pg/mL to about 500 pg/mL, preferably from about 50 pg/mL to about 250 pg/mL, more preferably from about 100 pg/mL to about 200 pg/mL. In one embodiment, said reference value is a sTREM-1 level, preferably a blood, plasma or serum level, ranging from about 20 pg/mL to about 500 pg/mL, preferably from about 50 pg/mL to about 250 pg/mL, more preferably from about 100 pg/mL to about 200 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, said reference value is a sTREM-1 level, preferably a blood, plasma or serum level, of about 50, 75, 100, 125, 150, 175, 200, 225 or 250 pg/mL. In one embodiment, said reference value is a sTREM-1 level, preferably a blood, plasma or serum level, of about 50, 75, 100, 125, 150, 175, 200, 225 or 250 pg/mL, as determined using an ELISA, or a corresponding TREM-1 level, in particular a corresponding sTREM-1 level, preferably a blood, plasma or serum level, as determined using another immunoassay, in particular an ECLIA. In one embodiment, said reference value is a sTREM-1 level, preferably a blood, plasma or serum level, of about 50, 55, 60, 65, 70, 75, 80, 85, 90, 95, 100, 105, 110, 115, 120, 125, 130, 135, 140, 145, or 150 pg/mL. In one embodiment, said reference value is a sTREM-1 level, preferably a blood, plasma or serum level, of about 50, 55, 60, 65, 70, 75, 80, 85, 90, 95, 100, 105, 110, 115, 120, 125, 130, 135, 140, 145, or 150 pg/mL, as determined using an ELISA, or a corresponding TREM-1 level, in particular a corresponding sTREM-1 level, preferably a blood, plasma or serum level, as determined using another immunoassay, in particular an ECLIA. In one embodiment, said reference value is a sTREM-1 level, preferably a blood, plasma or serum level, of about 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 pg/mL. In one embodiment, said reference value is a sTREM-1 level, preferably a blood, plasma or serum level, of about 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value is derived from the measure of the TREM-1 level, in particular of sTREM-1 level in a biological sample from one or more subjects diagnosed or identified as suffering, or having suffered, from a disease caused by a coronavirus, in particular from COVID-19. In one embodiment, the reference value is a reference sTREM-1 level derived from a reference population of subjects diagnosed or identified as suffering, or having suffered, from a disease caused by a coronavirus, in particular from COVID-19.

In one embodiment, said reference value is a TREM-1 level, in particular a sTREM-1 level, preferably a blood, plasma or serum level, ranging from about from about 50 pg/mL to about 800 pg/mL, preferably from about 75 pg/mL to about 600 pg/mL, more preferably from about 100 pg/mL to about 400 pg/mL, even more preferably from about 130 pg/mL to about 400 pg/mL. In one embodiment, said reference value is a TREM-1 level, in particular a sTREM-1 level, preferably a blood, plasma or serum level, ranging from about from about 50 pg/mL to about 800 pg/mL, preferably from about 75 pg/mL to about 600 pg/mL, more preferably from about 100 pg/mL to about 400 pg/mL, even more preferably from about 130 pg/mL to about 400 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, said reference value is a TREM-1 level, in particular a sTREM-1 level, preferably a blood, plasma or serum level, ranging from about 50 pg/mL to about 800 pg/mL, preferably from about 75 pg/mL to about 600 pg/mL, more preferably from about 100 pg/mL to about 400 pg/mL, even more preferably from about 130 pg/mL to about 400 pg/mL. In one embodiment, said reference value is a TREM-1 level, in particular a sTREM-1 level, preferably a blood, plasma or serum level, ranging from about 50 pg/mL to about 800 pg/mL, preferably from about 75 pg/mL to about 600 pg/mL, more preferably from about 100 pg/mL to about 400 pg/mL, even more preferably from about 130 pg/mL to about 400 pg/mL, as determined using an ELISA, or a corresponding TREM-1 level, in particular a corresponding sTREM-1 level, preferably a blood, plasma or serum level, as determined using another immunoassay, in particular an ECLIA. In one embodiment, said reference value is a TREM-1 level, in particular a sTREM-1 level, preferably a blood, plasma or serum level, ranging from about 130 pg/mL to about 600 pg/mL, preferably from about 200 pg/mL to about 500 pg/mL, more preferably from about 250 pg/mL to about 400 pg/mL, even more preferably from about 300 pg/mL to about 375 pg/mL. In one embodiment, said reference value is a TREM-1 level, in particular a sTREM-1 level, preferably a blood, plasma or serum level, ranging from about 130 pg/mL to about 600 pg/mL, preferably from about 200 pg/mL to about 500 pg/mL, more preferably from about 250 pg/mL to about 400 pg/mL, even more preferably from about 300 pg/mL to about 375 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, said reference value is a TREM-1 level, in particular a sTREM-1 level, preferably a blood, plasma or serum level, of about 75, 100, 125, 150, 175, 200, 225, 250, 275, 300, 325, 350, 375 or 400 pg/mL. In one embodiment, said reference value is a TREM-1 level, in particular a sTREM-1 level, preferably a blood, plasma or serum level, of about 75, 100, 125, 150, 175, 200, 225, 250, 275, 300, 325, 350, 375 or 400 pg/mL, as determined using an ELISA, or a corresponding TREM-1 level, in particular a corresponding sTREM-1 level, preferably a blood, plasma or serum level, as determined using another immunoassay, in particular an ECLIA. In one embodiment, said reference value is a TREM-1 level, in particular a sTREM-1 level, preferably a blood, plasma or serum level, of about 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, or 430 pg/mL. In one embodiment, said reference value is a TREM-1 level, in particular a sTREM-1 level, preferably a blood, plasma or serum level, of about 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, or 430 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, said reference value is a TREM-1 level, in particular a sTREM-1 level, preferably a blood, plasma or serum level, of about 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 pg/mL. In one embodiment, said reference value is a TREM-1 level, in particular a sTREM-1 level, preferably a blood, plasma or serum level, of about 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the therapy is an immunomodulatory therapy or anti-inflammatory therapy.

Examples of immunomodulatory therapies or anti-inflammatory therapies include, without being limited to, corticosteroids, checkpoint inhibitors such as anti-PD-1, anti-PD-L1 and anti-CTLA4; TLR (Toll-like receptors) inhibitors; cytokine inhibitors such as anti-cytokine (for example anti-IL-6 agents) or anti-cytokine receptors (for example IL-1RA for interleukin 1 receptor antagonist); G-CSF (granulocyte-colony stimulating factor); IL-7 (interleukin-7); inhibitors of immunostimulants such as CD28 antagonist peptides and antibodies, in particular monoclonal antibodies, against CD28; cellular therapies such as adoptive cell therapies; and TREM-1 inhibitors.

In one embodiment, the immunomodulatory therapy or anti-inflammatory therapy is selected from the group comprising or consisting of corticosteroids, checkpoint inhibitors; TLR (Toll-like receptors) inhibitors; cytokine inhibitors; G-CSF; IL-7; inhibitors of immunostimulants; cellular therapies; and TREM-1 inhibitors.

In one embodiment, the therapy is a TREM-1 inhibitor as described hereinabove. In one embodiment, the therapy is a TLT-1 peptide as described hereinabove, such as a TLT-1 peptide, preferably of 12, 13, 14, 15, 16, 17, 18, 19, or 20 amino acids, comprising or having an amino acid sequence as set forth in SEQ ID NO: 10 or a sequence having at least 60, 65, 70, 75, 80, 85, 90, 95, 96, 97, 98 or 99% identity with SEQ ID NO: 10.

In one embodiment, the present invention relates to an in vitro method for identifying a subject suffering from a disease caused by a coronavirus, in particular COVID-19, susceptible to respond to a therapy, preferably a TREM-1 inhibitor as described hereinabove, said method comprising:
measuring the level of soluble TREM-1 (sTREM-1) in a biological sample from the subject; and
comparing the level of sTREM-1 measured in the biological sample from the subject to a reference value, preferably a reference sTREM-1 level derived from a reference population of subjects diagnosed or identified as suffering, or having suffered, from a disease caused by a coronavirus, in particular from COVID-19,
   wherein a level of sTREM-1 measured in the biological sample from the subject which is higher than the reference value as described hereinabove indicates that the subject suffering from a disease caused by a coronavirus, in particular COVID-19, is susceptible to respond to a therapy, preferably a TREM-1 inhibitor as described hereinabove.

The present invention also relates to a method for treating a subject suffering from a disease caused by a coronavirus, in particular COVID-19, identified as being susceptible to respond to a therapy as described hereinabove, preferably a TREM-1 inhibitor as described hereinabove, said method comprising:
identifying a subject suffering from a disease caused by a coronavirus, in particular COVID-19, as being susceptible to respond to a therapy, preferably a TREM-1 inhibitor, as described hereinabove; and
treating the subject suffering from a disease caused by a coronavirus, in particular COVID-19, identified as being susceptible to respond to a therapy, preferably a TREM-1 inhibitor as described hereinabove, by administering to said subject said therapy, preferably said TREM-1 inhibitor as described hereinabove.

Another object of the invention is an in vitro method for monitoring the effectiveness of a therapy administered to a subject suffering from a disease caused by a coronavirus as described hereinabove, said method comprising:
measuring the level of TREM-1, in particular of sTREM-1, in a biological sample from the subject as described hereinabove; and
comparing the level of TREM-1, in particular of sTREM-1, measured in the biological sample from the subject to a reference value, preferably to a personalized reference value of the subject.

In one embodiment, the present invention relates to an in vitro method for monitoring the effectiveness of a therapy administered to a subject suffering COVID-19, said method comprising:
measuring the level of sTREM-1 in a biological sample from the subject as described hereinabove; and
comparing the level of sTREM-1 measured in the biological sample from the subject to a reference value as described hereinabove, preferably to a personalized reference value of the subject.

In one embodiment, the present invention relates to an in vitro method for monitoring the effectiveness of a therapy administered to a subject suffering from a severe form and/or at least one complication of a disease caused by a coronavirus, in particular COVID-19, as described hereinabove.

In one embodiment, the therapy is an immunomodulatory therapy or anti-inflammatory therapy as described hereinabove.

In one embodiment, the immunomodulatory therapy or anti-inflammatory therapy is selected from the group comprising or consisting of corticosteroids, checkpoint inhibitors; TLR (Toll-like receptors) inhibitors; cytokine inhibitors; G-CSF; IL-7; inhibitors of immunostimulants; cellular therapies; and TREM-1 inhibitors.

In one embodiment, the therapy is a TREM-1 inhibitor as described hereinabove. In one embodiment, the therapy is a TLT-1 peptide as described hereinabove, in particular a TLT-1 peptide, preferably of 12, 13, 14, 15, 16, 17, 18, 19, or 20 amino acids, comprising or having an amino acid sequence as set forth in SEQ ID NO: 10 or a sequence having at least 60, 65, 70, 75, 80, 85, 90, 95, 96, 97, 98 or 99% identity with SEQ ID NO: 10.

In one embodiment, a level of TREM-1, in particular of sTREM-1, measured in the biological sample from the subject lower than the reference value as described hereinabove indicates that the therapy is effective in the subject.

Accordingly, in one embodiment, a level of TREM-1, in particular of sTREM-1, measured in the biological sample from the subject equal or higher than the reference value as described hereinabove indicates that the therapy is not effective in the subject.

In one embodiment, the level of TREM-1, in particular of sTREM-1, is measured at least 1, 2, 3, 4, 5, 6, 7, 8, 9, or 10 day(s) after the start of the therapy (or is measured in a biological sample obtained from the subject at least 1, 2, 3, 4, 5, 6, 7, 8, 9, or 10 day(s) after the start of the therapy). In one embodiment, the level of TREM-1, in particular of sTREM-1, is measured at least 1, 2, 3, 4, or 5 day(s) after the start of the therapy (or is measured in a biological sample obtained from the subject at least 1, 2, 3, 4, or 5 day(s) after the start of the therapy). In one embodiment, the level of TREM-1, in particular of sTREM-1, is measured 1, 2, 3, 4, 5, 6, 7, 8, 9, or 10 day(s) after the start of the therapy (or is measured in a biological sample obtained from the subject 1, 2, 3, 4, 5, 6, 7, 8, 9, or 10 day(s) after the start of the therapy). In one embodiment, the level of TREM-1, in particular of sTREM-1, is measured 1, 2, 3, 4, or 5 day(s) after the start of the therapy (or is measured in a biological sample obtained from the subject 1, 2, 3, 4, or 5 day(s) after the start of the therapy). In one embodiment, the level of TREM-1, in particular of sTREM-1, is measured 5 days after the start of the therapy (or is measured in a biological sample obtained from the subject 5 days after the start of the therapy).

In one embodiment, the level of TREM-1, in particular of sTREM-1, is measured in biological samples obtained from the subject at regular interval after the start of the therapy. In one embodiment, the level of TREM-1, in particular of sTREM-1, is measured in biological samples obtained from the subject every 2, 3, or 4 days after the start of the therapy.

In one embodiment, the reference value is a personalized reference value, i.e., the reference value is a TREM-1 level, in particular a sTREM-1 level, measured in a biological sample obtained from the subject. In one embodiment, the personalized reference value is a TREM-1 level, in particular a sTREM-1 level, measured in a biological sample obtained from the subject at baseline, i.e., a baseline TREM-1 or sTREM-1 level. In one embodiment, the baseline level is a TREM-1 or sTREM-1 level measured in a biological sample obtained from the subject before the start of medical care. In one embodiment, the baseline level is a TREM-1 or sTREM-1 level measured in a biological sample obtained from the subject before the administration of a therapy or at the beginning of the administration of a therapy.

In one embodiment, the present invention relates to an in vitro method for monitoring the effectiveness of a therapy, preferably a TREM-1 inhibitor, administered to a subject suffering from a disease caused by a coronavirus, in particular COVID-19, said method comprising:
measuring the level of sTREM-1 in a biological sample from the subject as described hereinabove; and
comparing the level of sTREM-1 measured in the biological sample from the subject to a reference value, preferably a personalized reference value such as a sTREM-1 level measured in a biological sample obtained from the subject at baseline,
   wherein a level of sTREM-1 measured in the biological sample from the subject lower than the reference value as described hereinabove indicates that the therapy is effective in the subject.

### BRIEF DESCRIPTION OF THE DRAWINGS

FIG. 1 is a Kaplan-Meier graph showing the survival over 10 days of transgenic mice expressing the human SARS-CoV-2 receptor (K18 ACE2) infected with SARS-CoV-2 at t₀ and administered by intraperitoneal injection with 2 mg of TLT-1 peptide LR12 or vehicle (NaCl 0.9%) at t₀+1 h, t₀+24 h, t₀+48 h, t₀+72 h and t₀+96 h. N=16 mice per treatment group. P value calculated by a Log rank test.

FIG. 2 is a graph showing the evolution of the bodyweight over 10 days of transgenic mice expressing the human SARS-CoV-2 receptor (K18 ACE2) infected with SARS-CoV-2 at t₀ and administered by intraperitoneal injection with 2 mg of TLT-1 peptide LR12 or vehicle (NaCl 0.9%) at t₀+1 h, t₀+24 h, t₀+48 h, t₀+72 h and t₀+96 h. N=16 mice per treatment group. Data are represented as mean and SEM (standard error to the mean). The last observation carried forward (LOCF) approach was used to impute missing data. P value was calculated by a mixed-effect analysis.

FIG. 3 is a graph showing the bodyweight loss over 10 days of transgenic mice expressing the human SARS-CoV-2 receptor (K18 ACE2) infected with SARS-CoV-2 at t₀ and administered by intraperitoneal injection with 2 mg of TLT-1 peptide LR12 or vehicle (NaCl 0.9%) at t₀+1 h, t₀+24 h, t₀+48 h, t₀+72 h and t₀+96 h. N=16 mice per treatment group. Data are represented as mean and SEM (standard error to the mean). The last observation carried forward (LOCF) approach was used to impute missing data. P value was calculated by a mixed-effect analysis.

FIG. 4 is a graph showing the clinical score over 10 days of transgenic mice expressing the human SARS-CoV-2 receptor (K18 ACE2) infected with SARS-CoV-2 at t₀ and administered by intraperitoneal injection with 2 mg of TLT-1 peptide LR12 or vehicle (NaCl 0.9%) at t₀+1 h, t₀+24 h, t₀+48 h, t₀+72 h and t₀+96 h. The clinical score was assessed on a scale of 1 (healthy mouse) to 5 (dead mouse), with a lower score corresponding to a better clinical status. N=16 mice per treatment group. Data are represented as mean and SEM (standard error to the mean). P value was calculated by a mixed-effect analysis.

FIG. 5 is a graph showing the respiratory score over 10 days of transgenic mice expressing the human SARS-CoV-2 receptor (K18 ACE2) infected with SARS-CoV-2 at t₀ and administered by intraperitoneal injection with 2 mg of TLT-1 peptide LR12 or vehicle (NaCl 0.9%) at t₀+1 h, t₀+24 h, t₀+48 h, t₀+72 h and t₀+96 h. The respiratory score was assessed on a scale of 0 (normal, rapid mouse respiration) to 5 (dead mouse), with a lower score corresponding to a better respiratory function. N=16 mice per treatment group. Data are represented as mean and SEM (standard error to the mean). P value was calculated by a mixed-effect analysis.

FIG. 6 is a graph showing sTREM-1 plasma concentration over time for groups 1-4 (at 24 and 72 hours after infection for groups 1 and 2, and at 48 and 96 hours after infection for groups 3 and 4) of transgenic mice expressing the human SARS-CoV-2 receptor (K18 ACE2) infected with SARS-CoV-2 at t₀ and administered by intraperitoneal injection with 2 mg of TLT-1 peptide LR12 or vehicle (NaCl 0.9%) at t₀+1 h, t₀+24 h, t₀+48 h, t₀+72 h and t₀+96 h. N=8 mice per group. Data are represented as box and whiskers plot from min to max and showing all points. p values were calculated with a non-parametric t-test comparing LR12-treated and vehicle-treated conditions.

FIGS. 7A-J are a set of graphs showing the plasma concentrations over time for groups 1-4 (at 24 and 72 hours after infection for groups 1 and 2, and at 48 and 96 hours after infection for groups 3 and 4) of the following cytokines/chemokines: interferon γ or IFNγ (FIG. 7A), keratinocyte chemoattractant or KC (FIG. 7B), monocyte chemoattractant protein 1 or MCP-1 (FIG. 7C), interleukin-12 (active heterodimer) or IL12p70 (FIG. 7D), Regulated on Activation, Normal T cell Expressed and Secreted or RANTES (FIG. 7E), interferon gamma-induced protein 10 or IP-10 (FIG. 7F), interleukin-10 or IL-10 (FIG. 7G), interferon α or IFN-α (FIG. 7H), interleukin-6 or IL-6 (FIG. 7I), and granulocyte-macrophage colony-stimulating factor or GM-CSF (FIG. 7J) of transgenic mice expressing the human SARS-CoV-2 receptor (K18 ACE2) infected with SARS-CoV-2 at t₀ and administered by intraperitoneal injection with 2 mg of TLT-1 peptide LR12 or vehicle (NaCl 0.9%) at t₀+1 h, t₀+24 h, t₀+48 h, t₀+72 h and t₀+96 h. N=8 mice per group. Data are represented as box and whiskers plot from min to max and showing all points. p values were calculated with a non-parametric t-test comparing LR12-treated and vehicle-treated conditions at different timepoints.

FIGS. 8A-O are a set of graphs showing the number of CD45⁺ cells (FIG. 8A), eosinophils (FIG. 8B), monocytes-derived macrophages (FIG. 8C), interstitial macrophages (FIG. 8D), alveolar macrophages (FIG. 8E), neutrophils (FIG. 8F), CD103⁺ dendritic cells (FIG. 8G), monocytes-derived dendritic cells (FIG. 8H), conventional dendritic cells (FIG. 8I), CD11b⁺ dendritic cells (FIG. 8J), NK cells (FIG. 8K), CD8⁺ lymphocytes (FIG. 8L), CD4⁺ lymphocytes (FIG. 8M), NKT cells (FIG. 8N), plasmacytoid dendritic cells (FIG. 8O) in lungs harvested 48 hours after infection from transgenic mice expressing the human SARS-CoV-2 receptor (K18 ACE2) infected with SARS-CoV-2 at t₀ and administered by intraperitoneal injection with 2 mg of TLT-1 peptide LR12 or vehicle (NaCl 0.9%) at t₀+1 h, t₀+24 h, and t₀+48 h. N=8 mice per group. Data are represented as mean and SEM (standard error to the mean). P values are calculated by unpaired parametric t-test.

### EXAMPLES

The present invention is further illustrated by the following examples.

### Example 1: Mouse Model of SARS-CoV-2 Infection

### Materials and Methods

### Material

Mice: transgenic male mice B6.Cg-Tg(K18-ACE2)2Prlmn/J, 7-week-old at arrival, were obtained from Charles River (The Jackson Laboratory). The transgenic B6.Cg-Tg(K18-ACE2)2Prlmn/J mice express the human SARS-CoV-2 receptor (angiotensin-converting enzyme 2 [hACE2]) under a cytokeratin 18 promoter (K18) and are susceptible to SARS-CoV-2 pulmonary infection (Yinda et al. K18-hACE2 mice develop respiratory disease resembling severe COVID-19. PLoS Pathog. 2021 Jan. 19; 17(1):e1009195). The animals were housed in ventilated and enriched plastic cages containing irradiated sawdust as a bedding material, as prescribed by the housing standards throughout the experimental phase. Mice were housed in groups of maximum 5 animals per cage on a regular light-dark cycle, 22±2° C. and at 50±10% relative humidity. During the acclimation phase and experimental phase, standard diet (RM1 (E) 801492, SDS) and tap water were provided ad libitum. All procedures performed on animals in the course of the study were reviewed and approved by the Institutional Animal Care and Use Committee (IACUC). All the in vivo protocol design and procedures were approved by an Ethical Committee under ethical protocol 2020101616517580_v1 #27729.

SARS-CoV-2 strain: SARS-CoV-2 was isolated from a patient with laboratory-confirmed COVID-19 in Toulouse, France. Compared to the sequence of the Wuhan reference strain, an alignment showed 99.96% identity between the two strains. One notable variation between the two strains is a glycine (Gly) at position 614 in Spike protein of the Toulouse strain, vs. an aspartic acid (Asp) at the same position in the Wuhan reference strain.

TLT-1 peptide: mouse LR12 (LQEEDTGEYGCV—SEQ ID NO: 20), the murine equivalent of human LR12 (LQEEDAGEYGCM—SEQ ID NO: 10), was provided as a −80° C. frozen stock solution at 40 mg/mL in a phosphate-citrate-arginine buffer. Solution for administration was extemporaneously prepared by thawing out an aliquot of stock solution at room temperature and by diluting it 4-times in physiological serum (NaCl 0.9%). Each mice will then receive 2 mg of peptide at each injection.

Vehicle: phosphate-citrate-arginine buffer was provided as vehicle, as a −80° C. frozen stock solution. Solution for administration was extemporaneously prepared by thawing out an aliquot of stock solution at room temperature and by diluting it 4-times in physiological serum (NaCl 0.9%).

### Methods

Infection: on day 0 at to, the mice were infected with 25 μL of DMEM (Dulbecco's Modified Eagle Medium) containing SARS-CoV-2 (SARS-CoV-2 strain isolated in Toulouse. Compared to the sequence of Wuhan reference strain, an alignment shows 99.96% identity between the two strains. One notable mutation is a GLY at position 614 in Spike protein, vs an ASP in Wuhan strain at the same position) through intranasal route (2.5×10³ PFU/mouse).

Treatment: following infection, the mice were administered by intraperitoneal (i.p.) injection either 200 μL of murine LR12 peptide (at a concentration of 10 mg/mL, i.e., 2 mg corresponding to a dose of 80 mg/kg for a mouse of 25 grams) or 200 μL of vehicle at t₀+1 h, t₀+24 h, t₀+48 h, t₀+72 h and t₀+96 h.

Mice were thus assigned to each of the following groups:
Group 1 (8 mice)—infection with 2.5×10³ PFU/mouse+vehicle: daily clinical signs and blood sampling at 24 and 72 hours;
Group 2 (8 mice)—infection with 2.5×10³ PFU/mouse+2 mg LR12: daily clinical signs and blood sampling at 24 and 72 hours;
Group 3 (8 mice)—infection with 2.5×10³ PFU/mouse+vehicle: daily clinical signs and blood sampling at 48 and 96 hours;
Group 4 (8 mice)—infection with 2.5×10³ PFU/mouse+2 mg LR12: daily clinical signs blood sampling at 48 and 96 hours;
Group 5 (8 mice)—infection with 2.5×10³ PFU/mouse+vehicle: lung harvest at 48 h; and
Group 6 (8 mice)—infection with 2.5×10³ PFU/mouse+2 mg LR12: lung harvest at 48 h.

Sample collection: for plasma production and cytokine analyzes, on day 1 (D1) and day 3 (D3), blood was collected from the mice of groups 1 and 2; and on day 2 (D2) and day 4 (D4), blood was collected from the mice of groups 3 and 4. Blood sampling was done before the treatment. On D2, lungs were harvested from the mice of groups 5-6 for flow cytometry analyzes.

Monitoring: from day 0 (D0) to day 10 (D10), the bodyweight and survival of all mice from groups 1 to 4 were monitored. From D0 to D10, the clinical score of the mice from groups 1 to 4 was recorded on a scale from 1 to 5 defined as follows: 1=healthy mouse; 2=mouse showing signs of malaise, including slight piloerection, slightly changed gait and increased ambulation; 3=mouse showing signs of strong piloerection, constricted abdomen, changed gait, periods of inactivity; 4=mouse with enhanced characteristics of the previous groups, but showing little activity and becoming moribund; 5=dead mouse. From D0 to D10, the clinical score of the mice from groups 1 to 4 was recorded on a scale from 0 to 5 defined as follows: 0=normal, rapid mouse respiration; 1=slightly decreased respiration; 2=moderately reduced respiration; 3=severely reduced respiration; 4=asphyxia; 5=dead mouse.

Flow cytometry analysis: lungs harvested at D2 from the mice of groups 5-6 were digested with collagenase and then filtered. Red blood cells were lysed using buffer and leukocytes were stained with the following antibodies: anti-CD45 (APC), anti-CD11b (VioGreen), anti-CD11c (VioBlue), anti-Siglec F (FITC), anti-CD64 (PE), anti-CD206 (PE Cy7), anti-I-Ab (PerCp Cy5.5), anti-Ly6C (AF700), anti-CD103 (APC Cy7), anti-Ly6G (BV711), anti-PDAC1 (BV605), anti-CD3 (FITC), anti-CD4 (VioGreen), anti-CD8 (PE), and anti-NK1.1 (PE Vio770). Cells were then fixed with PFA (paraformaldehyde) 4% before analysis with BD FACSAria Fusion device. The following cells populations were distinguished:

alveolar macrophages, interstitial macrophages, neutrophiles and recruited monocytes:
alveolar macrophages: Siglec F⁺ CD11b^{-/low} /CD64⁺CD11c⁺
interstitial macrophages: CD¹¹b⁺ I-Ab⁺ CD64^{int/high}
neutrophils: CD11b⁺Ly6G⁺
monocyte derived macrophages: CD11b⁺ I-Ab^{-/low} CD64⁺ Ly6C^{+/-}
   T cell subsets and NK cells:
CD4⁺ T cells: CD3⁺NK1.1⁻CD4⁺CD8⁻
CD8⁺ T cells: CD3⁺NK1.1⁻CD4⁻CD8⁺
NK cells: CD3⁻NK1.1⁺CD4⁻CD8⁻
   dendritic cell subsets and plasmacytoid cells:
CD11b+ dendritic cells: CD11b⁺ CD11c^{+/-} I-Ab⁺ CD64⁻
monocyte derived dendritic cells: CD11b⁺ I-Ab⁺ Ly6C⁺
CD103+ dendritic cells: CD103⁺ CD11c⁺ I-Ab⁺
conventional dendritic cells: CD11c⁺ CD11b⁻I-Ab⁺
plasmacytoid dendritic cells: CD11c⁺ PDCA1⁺I-Ab⁺

Cytokine measurement: cytokine levels were assessed in the blood samples collected on D1, D2, D3 and D4 from the mice of groups 1-4 (at D1 and D3 for groups 1 and 2, and at D2 and D4 for groups 3 and 4). sTREM1 was assessed by ELISA (mouse TREM-1 Quantikine ELISA kit, R&D Systems, R&D Systems, Bio-Techne). IL-10 (interleukin-10), KC (keratinocytes-derived chemokine also known as chemokine (C—X—C motif) ligand 1 or CXCL1), IL-6 (interleukin-6), IFNγ (interferon γ), IFNα (interferon α), IL-12p70 (interleukin-12 p70), MCP (monocyte chemoattractant protein-1 also known as chemokine ligand 2 or CCL2), RANTES (Regulated on Activation, Normal T cell Expressed and Secreted), IP-10 (interferon gamma-induced protein 10), and GM-CSF (granulocyte-macrophage colony-stimulating factor) were assessed with LEGENDplex™ Mouse Anti-Virus Response Panel (13-plex) with V-bottom Plate (Biolegend).

### Results

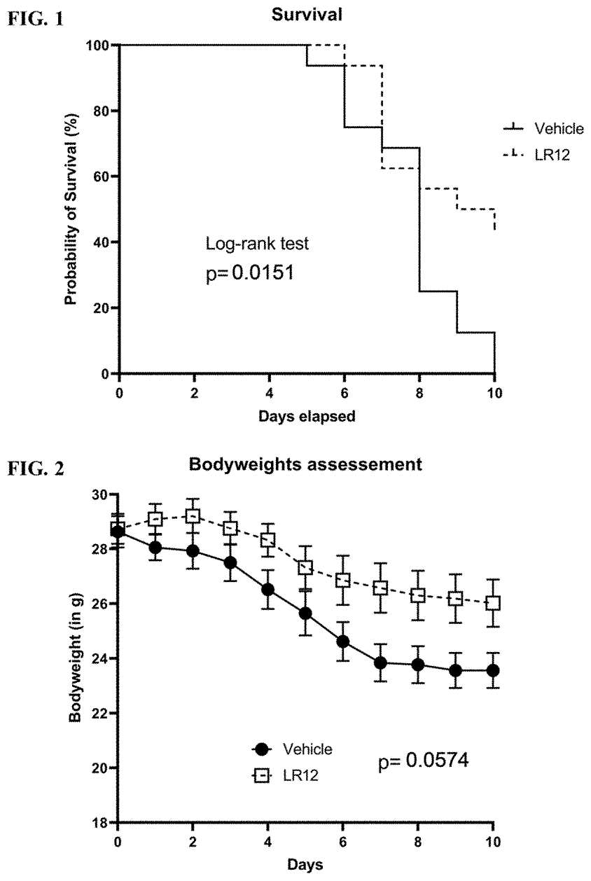

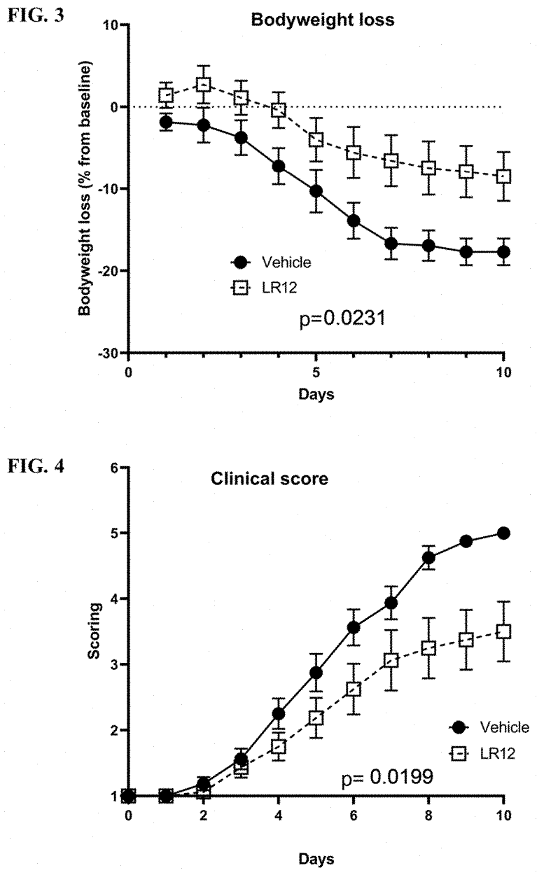

Mice from groups 1 to 4 were followed on a daily basis from D0 to D10 for survival. Results are reported in FIG. 1. A significant reduction in mortality was observed in LR12-treated mice as compared to vehicle-treated mice, thus confirming that LR12 displayed a protective effect against mortality in mice infected with SARS-CoV-2.

The body weight of each mouse from groups 1 to 4 was monitored on a daily basis from D0 to D10. Data from groups 1 and 3 (vehicle-treated mice) were pooled. Data from groups 2 and 4 (LR12-treated mice) were pooled. Bodyweight assessments are reported in FIG. 2. The percentage of bodyweight loss from day 0 to day 10 was calculated for each mouse and reported in FIG. 3. The last observation carried forward (LOCF) approach was used to impute missing data due to death of animals. The body weight of mice infected with SARS-CoV-2 decreased from D1 to D10, whether they were treated with LR12 or vehicle. However, the administration of LR12 (white squares) resulted in a significant reduced bodyweight loss compared to vehicle (black circles). This suggests that LR12 had a protective effect against the infection with SARS-CoV-2.

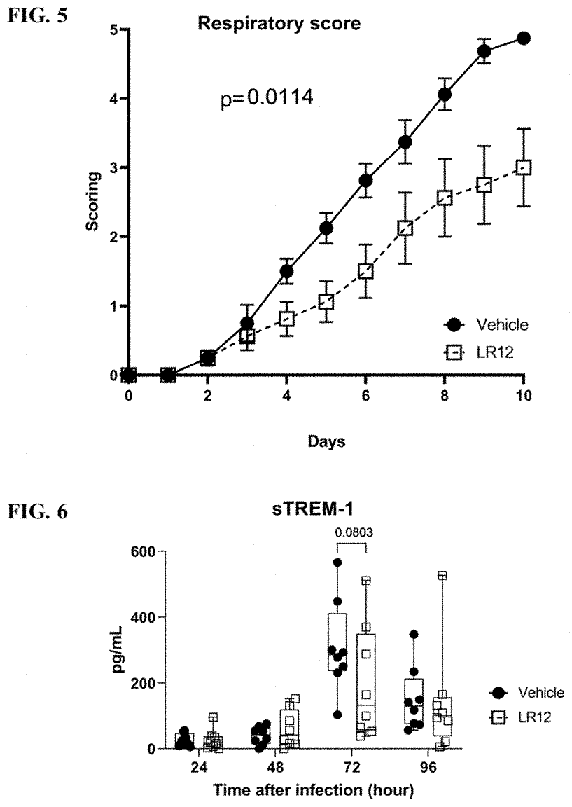

Mice were also monitored for welfare and behavior. The evolution of the clinical score over time showed an apparition of first clinical signs at day 2 and 3 in all groups, and a worsening up to day 10. However, the administration of LR12 resulted in less severe alterations of mice welfare as compared to vehicle, with a mean clinical score of 3.5 in the LR12-treated group and 5 in the vehicle-treated group (FIG. 4). The assessment of the respiratory function confirmed this protective effect of LR12. Indeed, the monitoring of the respiratory rate or capacity of all mice showed that signs of decreased respiratory capacity were observed at day 2 in all groups, and a worsening up to day 10 (FIG. 5). However, the administration of LR12 resulted in a significant less severe alteration of mice respiration as compared to vehicle, with a mean respiratory score of 3 in the LR12-treated group and 5 in the vehicle group. This confirms that LR12 displayed a strong protective effect against the infection with SARS-CoV-2 on both the evolution of clinical signs and respiratory function.

Soluble TREM-1 (sTREM-1) is a marker of the activation of the TREM-1 pathway. Measuring sTREM-1 thus reflects the level of activation of the receptor. sTREM-1 is therefore a useful marker for the monitoring of treatment effect with a TREM-1 inhibitor. Plasma sTREM-1 concentrations were measured at 24, 48, 72, and 96 hours following the infection, and data are represented in FIG. 6. Results show a time-dependent increase of sTREM-1 over time, peaking at 72 hours following the infection in both LR12-treated and vehicle-treated groups. However, LR12-treated mice showed a decrease in sTREM-1 levels at 72 hours post-infection from 286.1 pg/mL (median of vehicle-treated group) to 132.3 pg/mL (median of LR12-treated group), p=0.0803. This confirms that LR12 administration was associated with an inhibition of TREM-1 receptor activation.

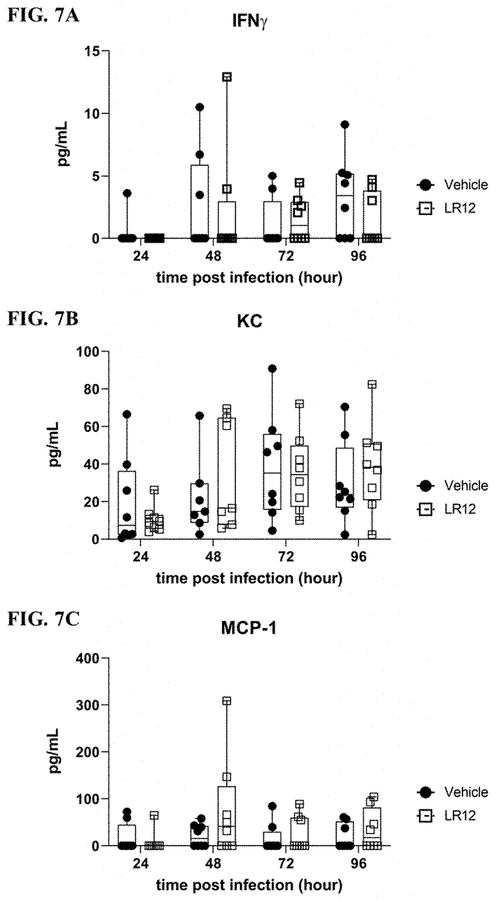

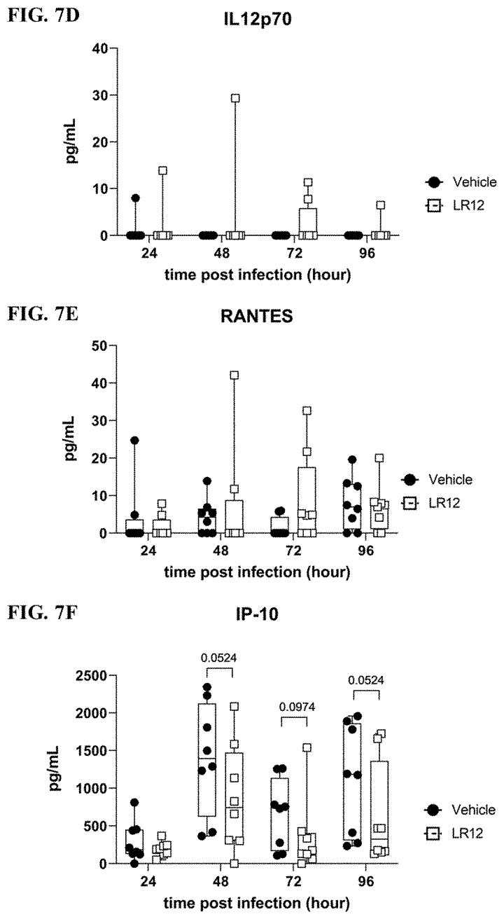

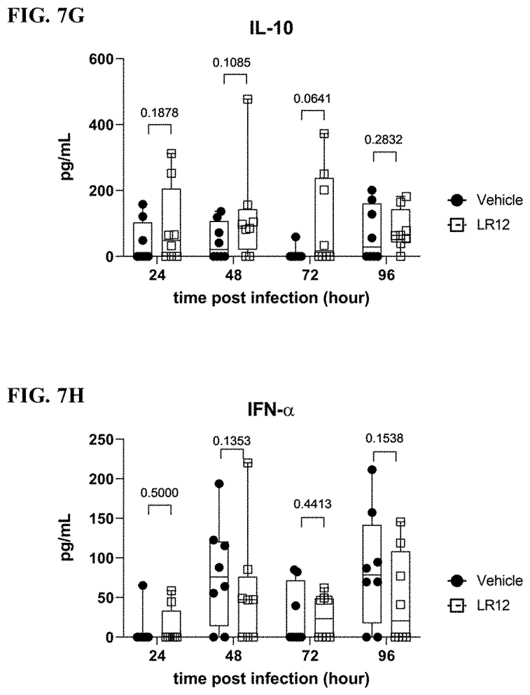

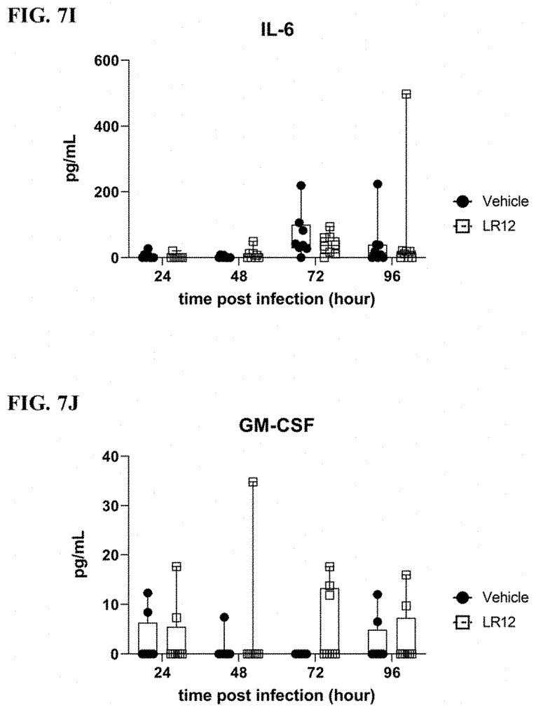

The evolution of the plasma concentration of several cytokines/chemokines: IFNγ (FIG. 7A), KC (FIG. 7B), MCP-1 (FIG. 7C), IL-12p70 (FIG. 7D), RANTES (FIG. 7E), IP-10 (CXCL10) (FIG. 7F), IL-10 (FIG. 7G), IFNα (FIG. 7H), IL-6 (FIG. 7I), and GM-CSF (FIG. 7J) was also assessed. Among these inflammatory markers, only IP-10, IL-10, IFNα, and IL-6 were significantly increased as compared to baseline values. Comparison of LR12-treated and vehicle-treated groups showed that LR12 was associated with a decrease in IP-10 and IFNα at all timepoints, a decrease of IL-6 at 72 hours, and an increase of IL-10 at 24 and 48 hours. This confirms that LR12 administration was associated with an immunomodulatory effect. IP-10 plays a role in the recruitment of several inflammatory cells such as monocytes/macrophages, neutrophils, T cells, NK cells and dendritic cells. IL-6 is a pleiotropic pro-inflammatory mediator, and high levels of IL-6 have been shown to be associated with severe forms of COVID-19. IL-10 is an anti-inflammatory mediator.

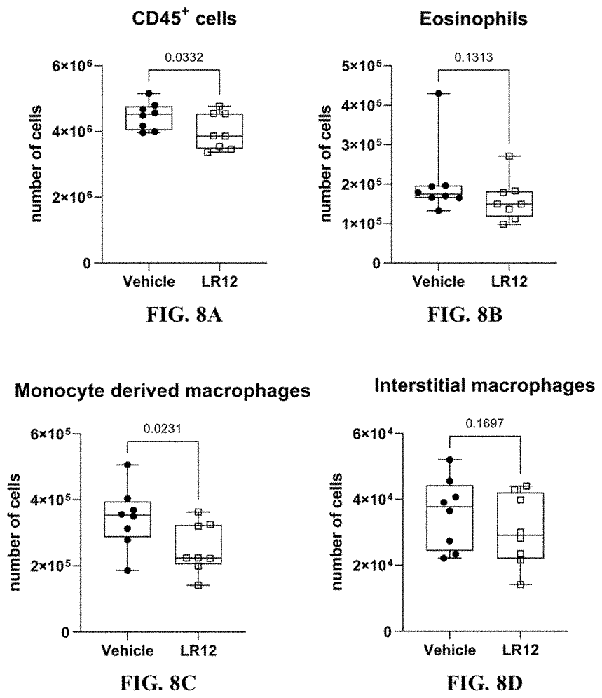

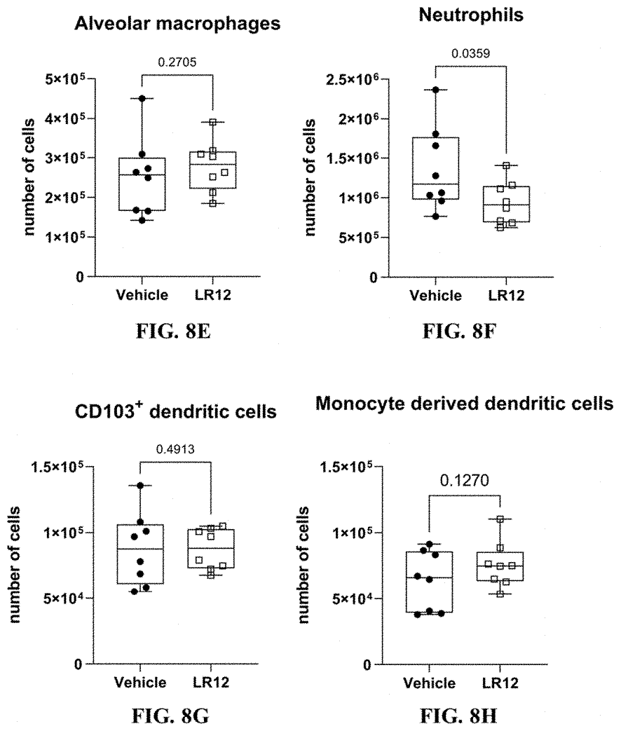

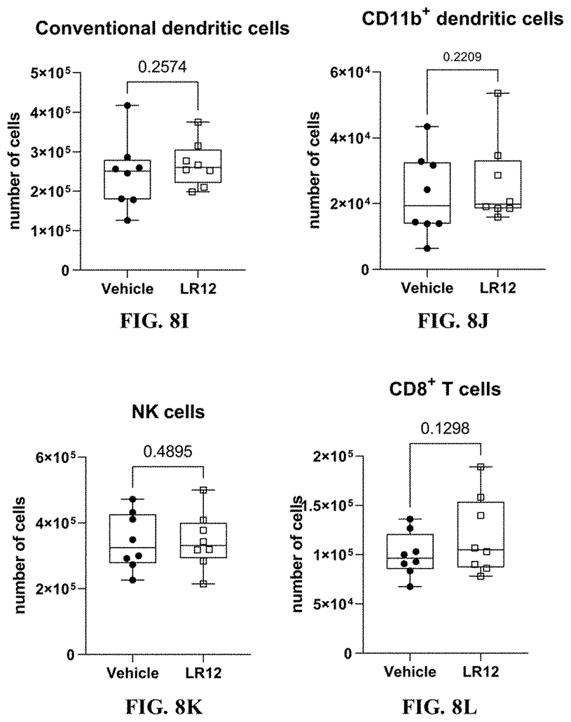

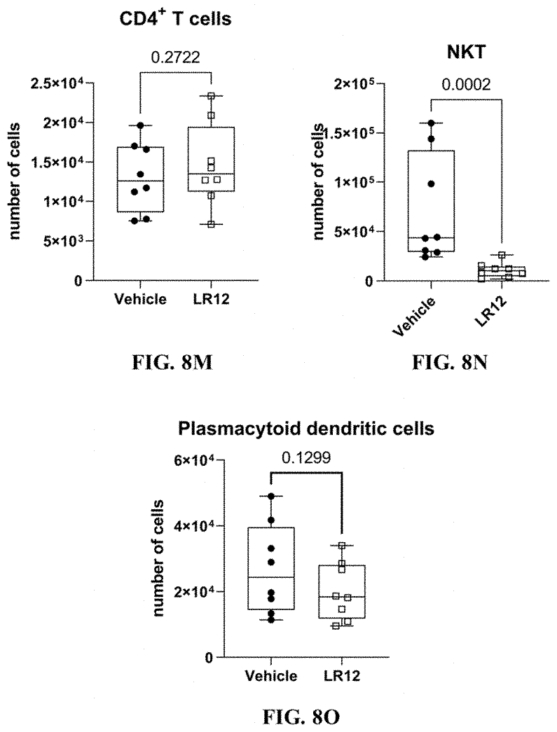

To further evaluate the effect of LR12 on the development of an immune response against SARS-Cov-2, the levels of various inflammatory cell-types were assessed in the lungs 48 hours post infection in LR-12-treated and vehicle-treated groups (FIGS. 8A-O). The total number of cells did not differ between both groups. A decrease in CD45⁺ cells was observed in the lungs of LR12-treated mice (FIG. 8A), which may be explained by a decrease in neutrophils and monocytes-derived macrophages. Indeed, the number of neutrophils (FIG. 8F) and monocytes-derived macrophages (FIG. 8C) in the lungs of LR12-treated mice were lower. LR12 had no effect on NK cells (FIG. 8K), CD8⁺ T cells (FIG. 8L), and CD4⁺ T cells (FIG. 8M). A lower number of NKT cells was also observed in the lungs of LR-12 treated mice (FIG. 8N). Although NKT cells contribute to the amplification of the antiviral immune response, these cells can also display increased expression of complement receptors and increased cytokine production, resulting in detrimental roles of NKT notably in the cytokine storm induction.
All these results confirm that TREM-1 inhibition with LR12 was associated with an immunomodulatory effect associated with a decreased inflammatory infiltrate, a decrease in the release of inflammatory mediators, which translated into a decrease in SARS-CoV-2 alteration of respiratory function and vital signs.

### Example 2: Clinical Trial

A randomized, double-blind, placebo-controlled study is described herein, aiming at evaluating the safety, tolerability and efficacy of nangibotide (TLT-1 peptide having an amino acid sequence as set forth in SEQ ID NO: 10, also known as LR12) in mechanically ventilated patients suffering from COVID-19.

The study is a randomized, double-blind, placebo-controlled, in which one dose of nangibotide will be tested versus placebo.

### Patient Randomization

A total of up to 730 patients suffering from COVID-19 and under mechanical ventilation (MV) are to be included in the study (including the 60 patients initially recruited). Randomization of patients is to be done in two stages to nangibotide or placebo. In stage one, 20 patients are randomized in a 1:1 ratio, in stage two, 40 patients are randomized in a 3:1 ratio to one of two treatment arms. The additional patients to be recruited will be randomized to nangibotide or placebo in a 1:1 ratio.

### Treatment

Patients receive a continuous intravenous (i.v.) infusion of nangibotide at 1.0 mg/kg/h or a matching placebo. Treatment with study drug must be initiated as early as possible but no later than 48 hours after the initiation of invasive mechanical ventilation. Patients are treated for 5 days or until discharge from critical care (i.e., intensive care unit), whichever is sooner. The treatment with study drug is in addition to standard of care. The duration of the study is 28 days. A follow-up visit is performed on day 8 and day 14. The end of study visit is at day 28. A further follow up visit will be undertaken on day 60.

### Inclusion Criteria

To be eligible for the study, patients must meet the following criteria:
Provided informed consent (emergency consent according to local regulations where approved);
Age 18 to 75 years (inclusive);
Invasive mechanical ventilation (respiratory support using a mechanical ventilator delivered via an endotracheal tube or tracheostomy) for acute respiratory failure caused by COVID-19 for less than 48 hours;
A PaO₂/FiO₂ ratio of <200 mmHg (<26.7 kPa); and
Confirmed laboratory diagnosis of COVID-19 within 7 days of meeting screening criteria.

### Exclusion Criteria

The presence of any of the following criteria excludes a patient from study enrolment:
Known pregnancy (positive urine or serum pregnancy test);
Currently receiving an immunomodulatory agent for the treatment of COVID-19 (including participation in clinical trials of such agents where treatment allocation is blinded or allocated on an open label basis);
Weight>95 kg;
Anticipated transfer to another hospital, which is not a study site within 72 hours;
Expected to die within 6 months of treatment due to underlying chronic disease; or
Limitations of care in place during current hospital admission.

### Criteria for Evaluation

### Primary Endpoint

The primary endpoint is the incidence of adverse events and mortality until day 28 and/or the clinical status (determined using the 7-point ordinal scale detailed below) assessed at day 28.

### Secondary Endpoints

Additional Safety Parameters:
Safety laboratory tests (as part of routine clinical care): hematology, coagulation, plasma biochemistry
Adverse events (AEs), serious adverse events (SAEs) and deaths
Suspected adverse drug reactions (serious and non-serious)

Efficacy Parameters:
Improvement of clinical status until end of study using a 7 ordinal scale as detailed below on all study days until day 14, day 28 and day 60:
   1—Not hospitalized, no limitations on activities
   2—Not hospitalized, limitation on activities;
   3—Hospitalized, not requiring supplemental oxygen;
   4—Hospitalized, requiring supplemental oxygen;
   5—Hospitalized, on non-invasive ventilation or high flow oxygen devices;
   6—Hospitalized, on invasive mechanical ventilation or ECMO;
   7—Death.
Mortality at day 28
PaO₂/FiO₂ ratio
Duration and nature of mechanical ventilation
Incidence of thromboembolic events
Incidence of secondary infections
Duration and nature of other organ support therapies
Functional status and mortality at day 60

Pharmacodynamics (exploratory):

sTREM-1, inflammatory exploratory biomarkers

### Statistical Methods

### Randomization and Stratification

In phase one, eligible patients are randomized in a 1:1 ratio of placebo or nangibotide into one of the two treatment arms. In phase two, eligible patients are randomized in a 1:3 ratio of placebo or nangibotide into one of the two treatment arms. The additional patients to be recruited will be randomized to nangibotide or placebo in a 1:1 ratio.

### Sample Size Determination

The sample size of this safety study has not been based on a formal sample size calculation. The initial sample size of 60 patients (20 treated with placebo, 40 treated with nangibotide) should support the identification of the most frequent adverse effects of nangibotide in this patient population. Additional patients, up to a total number of 730 (including the initial 60 patients) are to be recruited.

### Statistical Analyses

Primary Endpoint:

The primary endpoint is the incidence of adverse events and mortality until day 28. Usual descriptive statistics are to be used to analyze the safety parameters as follows:
Adverse events (AEs), serious adverse events (SAEs) and death
Safety laboratory tests: hematology, coagulation, plasma biochemistry

Secondary Endpoints:

### All-Cause Mortality at Day 28

The difference in death rates at day 28 is to be estimated along with an asymptotic and exact 95% confidence interval. In addition, the all-cause mortality at day 28 is to be analyzed in an exact logistic regression model adjusting for treatment and categorized baseline sTREM-1 level.

### Overall Survival

The Kaplan-Meier (KM) survival curves is to be provided with their 95% CI (confidence interval) for each group. A log-rank test is to be used to compare the treatment arms. In addition, a Proportional Hazard Cox model adjusting for treatment and categorized baseline sTREM-1 level is to be fitted to estimate the treatment effect expressed in terms of a hazard ratio with the 95% CI and p-value.

### Clinical Status

The clinical status is a 7-point ordinal scale which will be assessed at baseline (day 1) until day 14 and on day 28. A descriptive analysis of each category is to be performed by treatment group.
Distribution of the 7-point ordinal scale is to be compared between groups with a Cochran-Mantel Haenszel test using modified ridit scores.

1-14. (canceled)

15. A method for treating coronavirus disease 2019 (COVID-19) caused by a severe acute respiratory syndrome coronavirus 2 (SARS-CoV-2) in a subject in need thereof, said method comprising administering to the subject a triggering receptor expressed on myeloid cells-1 (TREM-1) inhibitor.

16. The method according to claim 15, wherein the subject is suffering from a severe form and/or at least one complication of COVID-19.

17. The method according to claim 16, wherein the at least one complication of COVID-19 is selected from the group consisting of respiratory failure, including acute respiratory failure or acute respiratory distress syndrome (ARDS); persistence of respiratory failure including the requirement for prolonged mechanical ventilation or failed extubation; secondary infection or superinfection; thrombotic complications including venous and/or arterial thromboembolism; pulmonary embolism; cardiocirculatory failure (also referred to as cardiovascular failure); renal failure; liver failure; and any combinations thereof.

18. The method according to claim 15, wherein the TREM-1 inhibitor is administered by intravenous infusion at a dose ranging from about 0.1 mg/kg/h to about 3 mg/kg/h.

19. The method according to claim 15, wherein the TREM-1 inhibitor is administered by intravenous infusion at a dose ranging from about 0.3 mg/kg/h to about 1 mg/kg/h.

20. The method according to claim 15, wherein the TREM-1 inhibitor is selected from the group consisting of peptides inhibiting the function, activity and/or expression of TREM-1; antibodies directed to TREM-1, soluble TREM-1 (sTREM-1), TREM-1 ligand and/or sTREM-1 ligand; small molecules inhibiting the function, activity and/or expression of TREM-1; siRNAs directed to TREM-1; shRNAs directed to TREM-1; antisense oligonucleotide directed to TREM-1; ribozymes directed to TREM-1; and aptamers directed to TREM-1.

21. The method according to claim 15, wherein the TREM-1 inhibitor is a peptide inhibiting the function, activity and/or expression of TREM-1 comprising an amino acid sequence selected from the group consisting of SEQ ID NO: 9, SEQ ID NO: 10, SEQ ID NO: 11, SEQ ID NO: 12, and SEQ ID NO: 13, or comprising an amino acid sequence with at least 80% identity with SEQ ID NO: 9, SEQ ID NO: 10, SEQ ID NO: 11, SEQ ID NO: 12, or SEQ ID NO: 13.

22. The method according to claim 15, wherein the TREM-1 inhibitor is a peptide inhibiting the function, activity and/or expression of TREM-1 comprising an amino acid sequence as set forth in SEQ ID NO: 10 or comprising an amino acid sequence with at least 80% identity with SEQ ID NO:10.

23. An in vitro method for identifying a subject suffering from coronavirus disease 2019 (COVID-19) caused by a severe acute respiratory syndrome coronavirus 2 (SARS-CoV-2) susceptible to respond to a triggering receptor expressed on myeloid cells-1 (TREM-1) inhibitor, said method comprising:
measuring the level of soluble TREM-1 (sTREM-1) in a biological sample from the subject; and
comparing the level of sTREM-1 measured in the biological sample from the subject to a reference value.

24. The in vitro method according to claim 23, wherein the subject is suffering from a severe form and/or at least one complication of COVID-19.

25. The method according to claim 15, wherein the subject to be treated is identified according to an in vitro method for identifying a subject suffering from coronavirus disease 2019 (COVID-19) caused by a severe acute respiratory syndrome coronavirus 2 (SARS-CoV-2) susceptible to respond to a triggering receptor expressed on myeloid cells-1 (TREM-1) inhibitor, said method comprising:
measuring the level of soluble TREM-1 (sTREM-1) in a biological sample from the subject; and
comparing the level of sTREM-1 measured in the biological sample from the subject to a reference value

26. An in vitro method for monitoring the effectiveness of a triggering receptor expressed on myeloid cells-1 (TREM-1) inhibitor administered to a subject suffering from coronavirus disease 2019 (COVID-19) caused by a severe acute respiratory syndrome coronavirus 2 (SARS-CoV-2), said method comprising:
measuring the level of soluble triggering receptor expressed on myeloid cells-1 (sTREM-1) in a biological sample from the subject; and
comparing the level of sTREM-1 measured in the biological sample from the subject to a reference value.

27. The in vitro method according to claim 26, wherein the reference value is a personalized reference value of the subject.

28. The in vitro method according to claim 27, wherein the personalized reference value of the subject is the level of sTREM-1 measured in a sample obtained from the subject before or at the beginning of the administration of the TREM-1 inhibitor.

29. The in vitro method according to claim 26, wherein the subject is suffering from a severe form and/or at least one complication of COVID-19.

30. The in vitro method according to claim 26, wherein the TREM-1 inhibitor is a peptide inhibiting the function, activity and/or expression of TREM-1 comprising an amino acid sequence selected from the group consisting of SEQ ID NO: 9, SEQ ID NO: 10, SEQ ID NO: 11, SEQ ID NO: 12, and SEQ ID NO: 13, or comprising an amino acid sequence with at least 80% identity with SEQ ID NO: 9, SEQ ID NO: 10, SEQ ID NO: 11, SEQ ID NO: 12, or SEQ ID NO: 13.


SEQUENCE LISTING (21 sequences)

SEQ ID NO: 1 - protein, 234 aa, Homo sapiens
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFNIVILLAGGFLSKSLVFSVLFAVTLRSFVP

SEQ ID NO: 2 - protein, 150 aa, Homo sapiens
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFRCSTLSFSWLVDS

SEQ ID NO: 3 - protein, 225 aa, Homo sapiens
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRYSFQVPGPLVWTLSPLFPSLCAERM

SEQ ID NO: 4 - protein, 250 aa, Homo sapiens
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIREKSMTFGIRRLDVESHPLPPLHTGHFRISQFFSQAGTQSLHSCYKGKPTP

SEQ ID NO: 5 - protein, 185 aa, Homo sapiens
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFN

SEQ ID NO: 6 - protein, 175 aa, Homo sapiens
LKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFN

SEQ ID NO: 7 - protein, 107 aa, Homo sapiens
LKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGF

SEQ ID NO: 8 - protein, 311 aa, Homo sapiens
MGLTLLLLLLLGLEGQGIVGSLPEVLQAPVGSSILVQCHYRLQDVKAQKVWCRFLPEGCQPLVSSAVDRRAPAGRRTFLTDLGGGLLQVEMVTLQEEDAGEYGCMVDGARGPQILHRVSLNILPPEEEEETHKIGSLAENAFSDPAGSANPLEPSQDEKSIPLIWGAVLLVGLLVAAVVLFAVMAKRKQGNRLGVCGRFLSSRVSGMNPSSVVHHVSDSGPAAELPLDVPHIRLDSPPSFDNTTYTSLPLDSPSGKPSLPAPSSLPPLPPKVLVCSKPVTYATVIFPGGNKGGGTSCGPAQNPPNNQTPSS

SEQ ID NO: 9 - protein, 17 aa, Artificial Sequence
  TLT-1 peptide
LQEEDAGEYGCMVDGAR

SEQ ID NO: 10 - protein, 12 aa, Artificial Sequence
  TLT-1 peptide
LQEEDAGEYGCM

SEQ ID NO: 11 - protein, 6 aa, Artificial Sequence
  TLT-1
LQEEDA

SEQ ID NO: 12 - protein, 6 aa, Artificial Sequence
  TLT-1 peptide
EDAGEY

SEQ ID NO: 13 - protein, 6 aa, Artificial Sequence
  TLT-1 peptide
GEYGCM

SEQ ID NO: 14 - DNA, 18 bp, Artificial Sequence
  Primer
gtggtgaccaaggggttc

SEQ ID NO: 15 - DNA, 20 bp, Artificial Sequence
  Primer
agatggatgtggctggaagt

SEQ ID NO: 16 - DNA, 20 bp, Artificial Sequence
  Primer
gtgaccaagggtttttcagg

SEQ ID NO: 17 - DNA, 20 bp, Artificial Sequence
  Primer
acaccggaaccctgatgata

SEQ ID NO: 18 - DNA, 19 bp, Artificial Sequence
  Primer
aaaggcaagaacgcctgac

SEQ ID NO: 19 - DNA, 20 bp, Artificial Sequence
  Primer
gggactttaccaagagggac

SEQ ID NO: 20 - protein, 12 aa, Artificial Sequence
  TLT-1 peptide
LQEEDTGEYGCV

SEQ ID NO: 21 - protein, 1273 aa, SARS coronavirus
  Spike glycoprotein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT